(19) United States
(12) Patent Application Publication
(10) Pub. No.: US 2007/0009989 A1
(43) Pub. Date: Jan. 11, 2007

(54) Synthetic heparanase molecules and uses thereof

(76) Inventors: Christian Steinkuhler, Promezia (IT); Armin Lahm, Promezia (IT); Michele Pallaoro, Promezia (IT); Caterina Nardella, Promezia (IT)

Correspondence Address: MERCK AND CO., INC, P O BOX 2000, RAHWAY, NJ, 07065-0907, US

(21) Appl. No.: 10/572,796

(22) PCT Filed: Sep. 17, 2004
(86) PCT No.: PCT/EP04/10517

§ 371 Date: Mar. 21, 2006

Related U.S. Application Data
(60) Provisional application No. 60/506,479, filed on Sep. 26, 2003.
(60) Provisional application No. 60/537,729, filed on Jan. 20, 2004.

(51) Int. Cl. C12P 21/06; C07H 21/04; C12N 9/24; C12N 1/18; C12N 5/06; C12N 15/74

(52) U.S. Cl. 435/69.1; 536/23.2; 435/200; 435/483; 435/254.2; 435/254.21; 435/254.23; 435/348

(57) ABSTRACT

The present invention relates to synthetically produced, enzymatically active heparanase nucleic acid molecules that are capable of expression in high yield heterologous expression systems, and to polypeptides encoded by said molecules. Also provided herein are methods of expressing mammalian heparanase in heterologous expression systems, wherein high yields of biologically active heparanase are produced compared to prior art methods.

### FIELD OF THE INVENTION

The present invention relates to synthetically produced, enzymatically active heparanase molecules that are capable of expression in high yield heterologous expression systems. Also provided herein are methods of expressing mammalian heparanase in heterologous expression systems.

### BACKGROUND OF THE INVENTION

Heparan sulfate proteoglycans (HSPGs) are ubiquitous macromolecules found in the extracellular matrix (ECM) and on the cell surface that contribute to the maintenance of cell-cell and cell-ECM interactions. HSPGs are composed of several heparan sulfate (HS) chains covalently linked to a protein core. Heparan sulfate facilitates binding of structural ECM proteins such as fibronectin, laminin, and collagen, to the cell surface and to other ECM proteins, suggesting roles for this glycosaminoglycan in self-assembly and insolubility of ECM components, in cell adhesion, and locomotion. Because of the importance of maintaining proper cell-cell and cell-ECM interactions, HSPGs play crucial structural and regulatory roles in the extracellular milieu, modulating important normal and pathological processes ranging from embryogenesis, morphogenesis and development to inflammation, angiogenesis and cancer metastasis.

In addition to the structural and cell-matrix anchoring roles mentioned above, the structural diversity of HS (Esko et al. J. Clin. Invest. 108:169-173 (2001); Tumbull et al. Trends Cell Biol. 11: 75-82 (2001)) allows HSPGs to interact with a variety of extracellular signaling proteins such as growth factors, enzymes, and chemokines. Growth factors such as fibroblast growth factors (FGF1 and FGF2), vascular endothelial growth factor (VEGF), hepatocyte growth factor, transforming growth factor β and platelet-derived growth factor, play important roles in tumor growth, invasiveness, and angiogenesis. In addition to acting as a depot for these signaling molecules, activating or stabilizing them, HSPGs may participate in ligand-receptor interactions, such as the binding of FGF2 to the diverse isoforms of the FGF receptor (Chang et al. FASEB J. 14: 137-144 (2000)).

Heparan sulfate is degraded by the endo β-D-glucuronidase heparanase, which is released by platelets, placental trophoblasts, and leukocytes. Heparanase specifically degrades heparan sulfate by cleaving the glycosidic bond through a hydrolase mechanism. This degradation results in the release of growth factors such as bFGF, urokinase plasminogen activator (uPA), and tissue plasminogen activator (tPA), which may either initiate neo-angiogenesis or potentiate ECM degradation. Additionally, HS cleavage by heparanase allows cells to migrate through the basal membranes (BM) and traverse the ECM barriers. HS degradation plays an important role in numerous physiological processes by allowing cells to quickly respond to extracellular changes. Therefore, inhibition of heparanase activity could affect pathologies correlated with altered cell migration, such as inflammation, metastasis, and autoimmune disorders.

Due to this pivotal role, heparanase is a potential novel target for the development of antitumor, antimetastasis, or anti-inflammatory drugs. For purposes of drug development, heparanase has a significant advantage over the matrix metalloproteases, which are also ECM-modifying enzymes, because it is likely a single gene product and not part of a complex family of related proteins. Exploiting heparanase as a drug target is presently hampered by both the scarcity of reliable high-throughput assays and by its complex biogenesis, which renders the production of large amounts of active protein a difficult task.

Human heparanase cDNA encodes a protein that is initially synthesized as a pre-pro-protein with a signal peptide sequence that is removed by signal peptidase upon translocation into the endoplasmic reticulum (ER). The resulting 65 kDa pro-form is further processed by removing the 157 N-terminal amino acids to yield the mature 50 kDa heparanase. The 50 kDa protein has a specific activity at least 100 fold higher than the unprocessed 65 kDa precursor (Vlodavsky et al. Nat. Med. 5: 793-802 (1999)). Interestingly, the 50 kDa protein is inactive if expressed as such in mammalian cells (Hulett et al. Nat. Med. 5: 803-809(1999)). It was proposed that the active form of the enzyme consists of a heterodimer between the 50 kDa fragment and an 8 kDa fragment arising from the excision of an intervening 6 kDa peptide by unidentified proteolytic enzyme(s) (Fairbanks et al. J. Biol. Chem. 274: 29587-29590 (1999)). Consistent with this hypothesis, McKenzie et al. (Biochem J 373: 423-435 (2003)) produced active heterodimeric heparanase in insect cells and confirmed that the 8 kDa subunit is necessary for heparanase activity.

Endogenous heparanase can be purified from various sources; however, low heparanase expression levels lead to the necessity for laborious and expensive purification procedures. For example, Toyoshima & Nakajima (J. Biol. Chem. 274: 24153-24160 (1999)) described a process for purifying endogenous human heparanase from platelets that requires four different chromatographic steps and lasts five days.

Another drawback to the purification of endogenous heparanase is that overall yields are characteristically low. For instance, Fairbanks et al. (J. Biol. Chem. 274, 29587-29590, (1999)) report the purification of only 22 μg of heparanase from platelets, with a yield of 6%. Similarly, Fuks and colleagues (U.S. Pat. No. 5,362,641) describe a 4000-fold purification of heparanase from 1.4 kg of protein derived from the human hepatoma cell line Sk-Hep-1, producing only 6.5 μg of purified heparanase protein with a yield of 1.9%. A 240,000-fold purification of heparanase from the same cell line was disclosed by Pecker et al. (U.S. Pat. No. 5,968,822); however, this process required over 500 liters of cell culture.

The identification and cloning of the human heparanase gene (Vlodavksy et al, Nature Med. 5: 793-802 (1999); Hulett et al, Nature Med. 5: 803-809 (1999); Toyoshima & Nakajima, J. Biol. Chem. 274: 24153-24160 (1999)) allowed the recombinant expression of heparanase protein in heterologous expression systems. However, serious deficiencies have been noted with such heterologous expression systems in relation to heparanase production. For example, Ben-Artzi et al. (WO 99/57244) describe the expression of recombinant human heparanase in bacterial, mammalian, yeast, and insect cells. Although heparanase expression was obtained, there was no detectable enzymatic activity associated with the recombinant protein when E. coli was host cell, and only the 70 kDa unprocessed precursor was detected when heparanase was expressed in the yeast Pichia pastoris.

Ben-Artzi and colleagues (supra) also describe the expression of recombinant heparanase in mammalian cells, namely, human kidney fibroblasts (293), baby hamster kidney cells (BHL21) and chinese hamster ovary cells (CHO). However, these expression systems are known to have low yields and high associated costs. Furthermore, despite the fact that processing of the recombinant full-length precursor to yield the active, mature protein is observed in these cells, no homogeneously processed protein is obtained because the processing reaction is inefficient. Additionally, the use of expression vectors driving the secretion of heparanase does not lead to production of recombinant heparanase in the conditioned medium of CHO cells, which have to be further stimulated to secrete heparanase by addition of calcium ionophore or PMA. Only a minor fraction of the secreted protein appeared to be correctly processed in this system.

The production of heparanase in insect cell expression systems such as Sf21 or High five cells is described in the art (WO 99/57244, WO 99/11798, U.S. Pat. No. 5,968,822; U.S. Pat. No. 6,348,344; and U.S. Pat. No. 6,190,875). However, although efficient secretion into the growth medium was observed with such methods, specific activity of the enzyme was very low and no correct processing was observed. For example, Ben-Artzi et al. (WO 99/57244) describe the introduction of protease cleavage sites downstream of positions 119 or 157 of the heparanase protein in order to generate a correctly processed heparanase in insect cell expression systems. However, these constructs were not shown to be enzymatically active.

McKenzie et al. (supra) described the production of active heterodimeric heparanase in insect cells. This system, however, has the disadvantage of requiring the simultaneous production of two different recombinant proteins (the 8 kDa and the 50 kDa subunits). Since admixture of the isolated 8 kDa and 50 kDa domains does not result in heparanase activation, the successful recovery of an active heterodimer by simultaneous expression probably relies on a co-translational formation of the heterodimeric complex. Treatment of this complex with glycanase leads to its dissociation and to the precipitation of the 50 kDa subunit, suggesting a poor stability and solubility.

Despite the methods described above to obtain heparanase in active or inactive form, it would be advantageous to produce biologically active heparanase molecules that are capable of expression in high yield, low cost heterologous expression systems. Said molecules can be used in inhibitor screening assays for the development of therapeutics or pharmaceuticals to inhibit and/or treat metastatic growth and/or inflammation.

### SUMMARY OF THE INVENTION

The present invention provides synthetic nucleic acid molecules that encode biologically active, mammalian heparanase, wherein the nucleic acid molecules are capable of expression in high yield heterologous expression systems. The synthetic heparanase molecules provided herein present a significant advance over wild-type heparanase, which is expressed at low levels in mammalian systems and improperly processed in heterologous expression systems. The synthetic molecules of the present invention can be used in inhibitor screening assays for the development of therapeutics or pharmaceuticals to inhibit and/or treat metastatic growth, autoimmune disorders, and/or inflammation.

In one aspect of the invention, the synthetic nucleic acid molecule described above comprises a sequence of nucleotides that encodes a mammalian heparanase protein, the sequence of nucleotides comprising two consensus cleavage sites recognized by an endoproteinase, the cleavage sites located between nucleotides encoding residues 100 and 168 of the heparanase protein. Said nucleic acid molecule encodes a heparanase protein which is capable of biological activity upon incubation with the appropriate enzyme.

This invention further relates to a synthetic mammalian heparanase nucleic acid molecule comprising a portion that encodes a mammalian heparanase protein, the protein coding portion consisting essentially of a sequence of nucleotides encoding an N-terminal fragment of about 8 kDa, a linker, and a sequence of nucleotides encoding a C-terminal fragment of about 50 kDa, wherein the N-terminal and C-terminal fragments encode protein fragments that are substantially similar to wild-type heparanase fragments and wherein the encoded mammalian heparanase protein is constitutively active.

Also provided herein are synthetically produced, biologically active, mammalian heparanase polypeptides and heparanase polypeptides comprising endoproteinase consensus cleavage sites that are capable of biological activity upon incubation with the appropriate enzyme.

The present invention further provides methods for expressing mammalian heparanase in heterologous expression systems, said methods resulting in high levels of biologically active heparanase expression.

As used throughout the specification and in the appended claims, the singular forms “a,” “an,” and “the” include the plural reference unless the context clearly dictates otherwise.

As used throughout the specification and appended claims, the following definitions and abbreviations apply:

A “conservative amino acid substitution” refers to the replacement of one amino acid residue by another, chemically similar, amino acid residue. Examples of such conservative substitutions are: substitution of one hydrophobic residue (isoleucine, leucine, valine, or methionine) for another; substitution of one polar residue for another polar residue of the same charge (e.g., arginine for lysine; glutamic acid for aspartic acid).

The term “mammalian” refers to any mammal, including a human being.

The term “treatment” refers to both therapeutic treatment and prophylactic or preventative measures. Those in need of treatment include those already with the disorder as well as those prone to have the disorder or those in which the disorder is to be prevented. A “disorder” is any condition that would benefit from treatment with molecules identified using the nucleic acid molecules and polypeptides described herein. Such disorders include, but are not limited to, cancer, inflammation and autoimmune disorders.

The term “vector” refers to some means by which DNA fragments can be introduced into a host organism or host tissue. There are various types of vectors including plasmid, virus (including adenovirus), bacteriophages and cosmids.

“Biologically active” refers to a protein having structural, regulatory, or biochemical functions attending a naturally occurring molecule or isoform thereof. In the context of heparanase, “biologically active” proteins comprise heparanase enzymatic activity.

“Substantially similar” means that a given sequence shares at least 80%, preferably 90%, more preferably 95%, and even more preferably 99% homology with a reference sequence. In the present invention, the reference sequence can be the full-length human heparanase nucleotide or amino acid sequence, or the nucleotide or amino acid sequence of the 8 kDa (SEQ ID NO:15) or 50 kDa (SEQ ID NO:16) heparanase fragments, as dictated by the context of the text. Thus, a heparanase protein sequence that is “substantially similar” to the 8 kDa human heparanase fragment (SEQ ID NO:15) will share at least 80% homology with the 8 kDa human heparanase fragment, preferably 90% homology, more preferably 95% homology and even more preferably 99% homology. Whether a given heparanase protein or nucleotide sequence is “substantially similar” to a reference sequence can be determined for example, by comparing sequence information using sequence analysis software such as the GAP computer program, version 6.0, available from the University of Wisconsin Genetics Computer Group (UWGCG). The GAP program utilizes the alignment method of Needleman and Wunsch (J. Mol. Biol. 48:443, 1970), as revised by Smith and Waterman (Adv. Appl. Math. 2:482, 1981).

A “gene” refers to a nucleic acid molecule whose nucleotide sequence codes for a polypeptide molecule. Genes may be uninterrupted sequences of nucleotides or they may include such intervening segments as introns, promoter regions, splicing sites and repetitive sequences. A gene can be either RNA or DNA. A preferred gene is one that encodes the invention peptide.

The term “nucleic acid” or “nucleic acid molecule” is intended for ribonucleic acid (RNA) or deoxyribonucleic acid (DNA), probes, oligonucleotides, fragment or portions thereof, and primers. DNA can be either complementary DNA (cDNA) or genomic DNA, e.g. a gene encoding the invention peptide.

“Wild-type heparanase” or “wild-type protein” or “wt protein” refers to a protein comprising a naturally occurring sequence of amino acids or variant thereof. The amino acid sequence of wild-type human heparanase is available in the art (Vlodavksy et al, Nature Med. 5: 793-802 (1999); Hulett et al, Nature Med. 5: 803-809 (1999); Toyoshima & Nakajima, J. Biol. Chem. 274(34): 24153-24160 (1999); which are herein incorporated by reference in their entirety).

“Wild-type heparanase gene” refers to a gene comprising a sequence of nucleotides that encodes a naturally occurring heparanase protein, including proteins of human origin or proteins obtained from another organism, including, but not limited to, insects such as Drosophila, amphibians such as Xenopus, and mammals such as rat, mouse and rhesus monkey. The nucleotide sequence of the human heparanase gene is available in the art (Genbank Accession No. AF155510; Toyoshima and Nakajima, supra, which are hereby incorporated by reference in their entirety).

“Substantially free from other proteins” or “substantially purified” means at least 90%, preferably 95%, more preferably 99%, and even more preferably 99.9%, free of other proteins. Thus, a heparanase protein preparation that is substantially free from other proteins will contain, as a percent of its total protein, no more than 10%, preferably no more than 5%, more preferably no more than 1%, and even more preferably no more than 0.1%, of non-heparanase proteins. Whether a given heparanase protein preparation is substantially free from other proteins can be determined by such conventional techniques of assessing protein purity as, e.g., sodium dodecyl sulfate polyacrylamide gel electrophoresis (SDS-PAGE) combined with appropriate detection methods, e.g., silver staining or immunoblotting.

### BRIEF DESCRIPTION OF THE DRAWINGS

FIG. 1 depicts the biosynthesis of human heparanase in mammalian cells.

FIG. 2, Panel A, shows a schematic view of the heparanase constructs with engineered TEV cleavage sites. Panel B (left) shows results of Western blot analysis of correctly processed wt heparanase expressed in COS7 cells (lane 1), hepTEV110 (lane 2), hepTEV110 after 16 hours incubation with (lane 3) or without (lane 4) 0.5 μM TEV protease, hepTEV110/158 (lane 5), hepTEV110/158 after 16 hours incubation with (lane 6) or without (lane 7) 0.5 μM TEV protease. Panel B (right) shows heparanase activity of hepTEV110 (column 1), hepTEV110 after 16 hours incubation with (column 2) or without (column 3) 0.5 μM TEV protease, hepTEV110/158 (column 4), hepTEV110/158 after 16 hours incubation with (column 5) or without (column 6) 0.5 μM TEV protease. Heparanase activity of these samples was assessed using the fluorimetric method.

FIG. 3: Panel A: Multiple sequence alignment of heparanase against related sequences. Predicted secondary structure elements are shown above the alignment (arrows=beta strands, cylinders=helix). The positions of the two cleavage sites are indicated by black triangles. The region of the excised heparanase segment substituted by the Hyaluronidase fragment is surrounded by a grey box. Panel B: Schematic view of the TIM barrel architecture. The location of the excised heparanase segment is indicated with the cleavage points shown as triangles. If present, the segment most likely obscures binding of the substrate (grey arrow) by beta/alpha units 1 and 2. Design of a shorter loop (dotted line) removes this constraint, leading to an active enzyme while, at the same time, maintaining the structural integrity of the enzyme.

FIG. 4: Panel A: schematic view of the single chain heparanase constructs described herein. Panel B, left: Western blot analysis of wt heparanase or single chain constructs expressed in COS7 cells. Bla is a control corresponding to the partially purified lysate of COS7 cells transfected only with a vector encoding for the reporter gene β-lactamase (see materials and methods section). Right: Heparanase activity of the same samples using the radiometric assay. Specific activity of all single chain constructs is normalized against that of the wt heparanase.

FIG. 5: Left, Western blot analysis of the correctly processed wt heparanase produced in COS7 cells or wt heparanase and single chain constructs expressed in Sf9 cells. Right: Heparanase activity of the same samples using the radiometric assay. Specific activity of wt heparanase and single chain constructs expressed in Sf9 cells is normalized against that of the correctly processed wt heparanase produced in COS7 cells.

FIG. 6: Size exclusion chromatography of FITC-HS degradation products obtained after incubation for 6 hours with hepGS3 (□) and hepHyal (▴) single chain proteins produced in insect cells compared to that of the correctly processed wt heparanase produced in COS7 cells (●) and to unprocessed FITC-HS (◯).

FIG. 7: Ionic strength dependence (panel A), inhibition by heparin (panel B) and pH-dependence (panel C) of wild-type heparanase produced in COS7 cells (●), hepGS3 (□) and hepHyal (▴) single chain constructs produced in insect cells using the fluorimetric activity assay. In the heparin titration experiment, the following IC50 values were obtained: hepwt, 0.9 ng/μl; hepGS3, 1.1 ng/μl; hepHyal, 1.5 ng/μl.

### DETAILED DESCRIPTION OF THE INVENTION

Heparanase is a mammalian enzyme that degrades heparan sulfate (HS) by cleaving the glycosidic bond through a hydrolase mechanism. HS degradation plays an important role in numerous physiological processes by allowing cells to quickly respond to extracellular changes by altering cell-cell and cell-ECM interactions. Because of the importance of these interactions, inhibition of heparanase activity could affect several pathologies such as tumor cell metastasis, T-cell mediated delayed type hypersensitivity, and autoimmunity.

Several lines of evidence suggest that heparanase is involved in tumor cell metastasis. First, expression levels of heparanase correlate with the metastatic potential of several tumors and tumor cell lines. Second, patients with aggressive metastatic disease have measurable heparanase activity in their urine. This observation is not seen with all cancer patients. Additionally, inhibition of heparanase activity by non-anticoagulant heparin derivatives reduced the incidence of metastases by B16 melanoma, Lewis lung carcinoma, and mammary adenocarcinoma cells. Finally, transfection of nonmetastatic murine cells with the human heparanase gene resulted in increased mortality and metastasis in two mouse models.

Human heparanase does not share substantial homology with any other known proteins. At the time of its discovery, evidence suggested that the heparanase gene was not a member of a gene family, but rather a single gene or at least the dominant endoglucuronidase involved in HSPG degradation. A second heparanase (hpa2), which shares 35% identity at the amino acid level, was later identified; however, hpa2 seems to serve a different function based on its tissue distribution. The absence of closely related proteins that accomplish analogous tasks, coupled with the above evidence demonstrating a role for heparanase in metastatic growth, make heparanase an excellent target for the development of therapeutics in these areas.

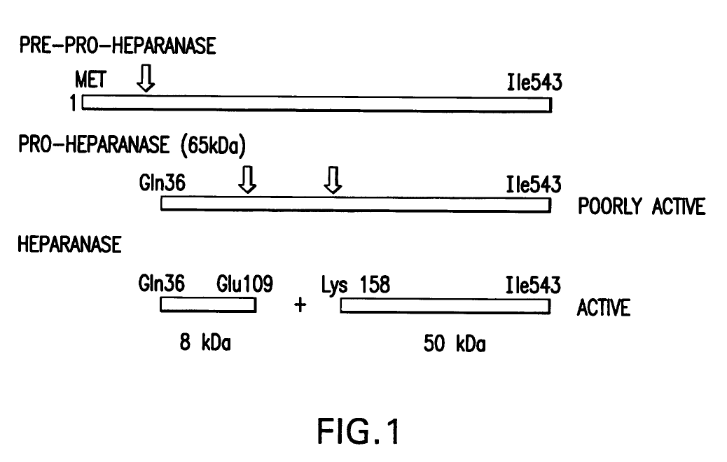

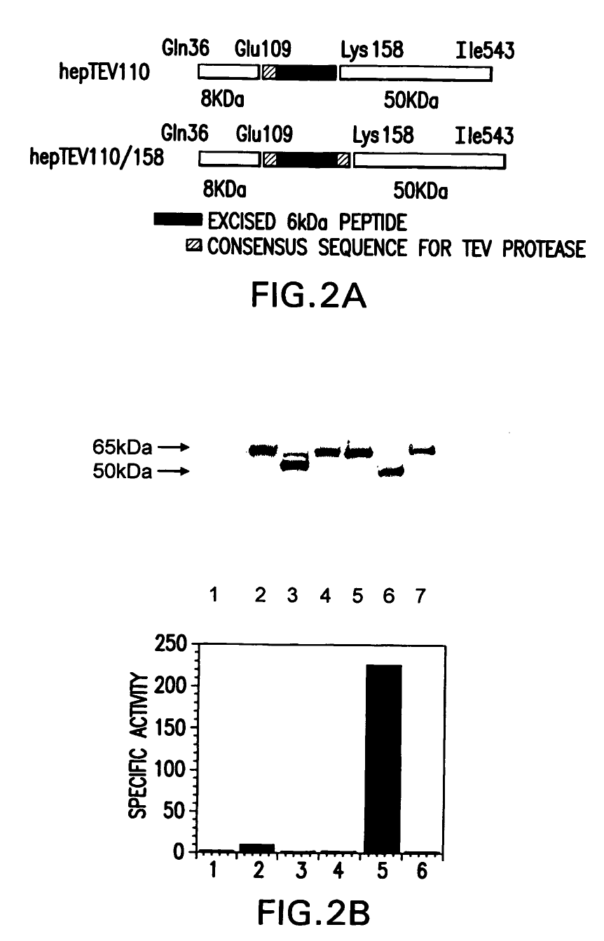

FIG. 1 depicts the biosynthesis of human heparanase. Briefly, the heparanase cDNA encodes a protein that is initially synthesized as a pre-pro-protein with a signal peptide sequence (residues Met¹-Ala³⁵) removed by signal peptidase upon translocation into the ER The resulting 65 kDa pro-form is further processed by removing the 157 N-terminal amino acids to yield the mature 50 kDa heparanase (SEQ ID NO:16). The 50 kDa protein has a specific activity at least 100 fold higher than the unprocessed 65 kDa precursor (Vlodavsky et al. Nat. Med. 5: 793-802 (1999)). The active form of the enzyme was proposed to be a heterodimer between the 50 kDa fragment and an 8 kDa fragment (SEQ ID NO:15) arising from the excision of an intervening 6 kDa peptide (residues Glu¹⁰⁹-Gln¹⁵⁷) by unidentified proteolytic enzyme(s) (hereinafter “intervening fragment” or “6 kDa fragment”) (Fairbanks et al. J. Biol. Chem. 274: 29587-29590 (1999).

Despite recent evidence showing that the 8 kDa subunit (SEQ ID NO:15) is necessary for heparanase activity (McKenzie et al. Biochem J. 373: 423-435 (2003)), the role of the 8 kDa subunit in the activation process of heparanase remained unclear prior to the studies disclosed herein: it could function as an essential subunit or, alternatively, act as a chaperone and be dispensable after having accomplished this function. It was also not clear whether other components besides the 8 kDa subunit are necessary to elicit heparanase activation.

Multiple sequence alignments and secondary structure prediction lead to a model of the human heparanase according to which the protein adopts a TIM barrel fold, as found in several glycosidases (Hulett et al. Biochemistry 39:15659-15667 (2000)). This common fold motif usually consists of 8 alternating α-helices and β-strands. Within the 50 kDa fragment clear homology is observed only starting with the 3^{rd} α/β unit of the TIM barrel fold, suggesting either that heparanase adopts a novel fold consisting of only 6α/β units or that other parts of the protein contribute the missing units. It was postulated that the 8-kDa fragment might contribute the missing structural elements (Hulett et al., supra).

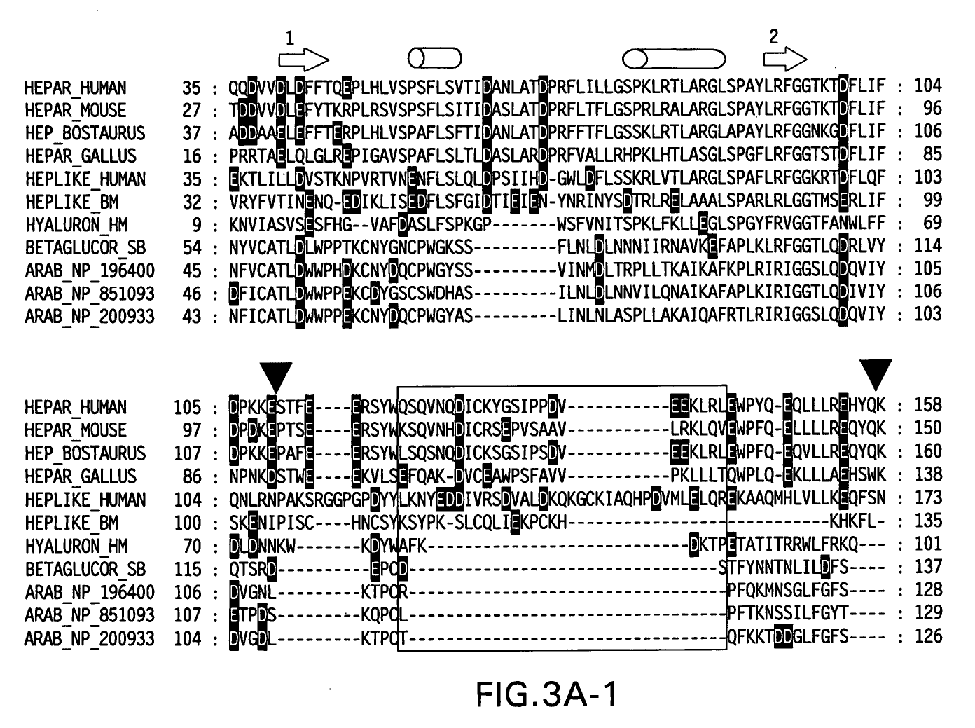

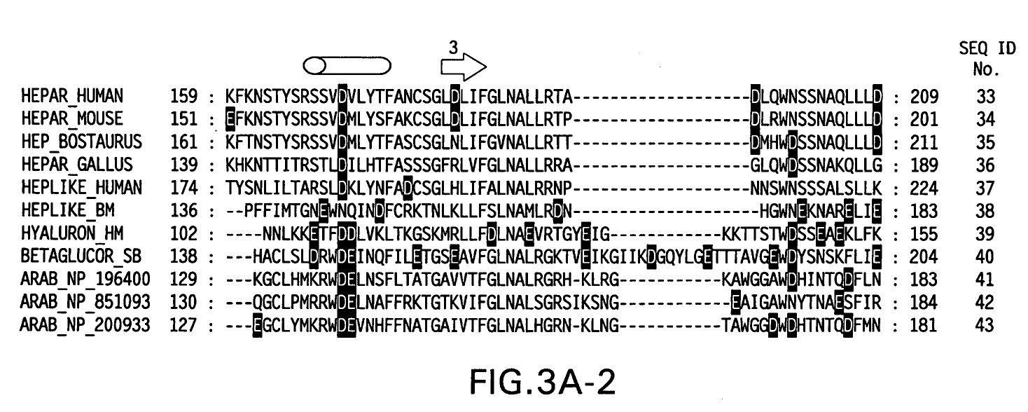

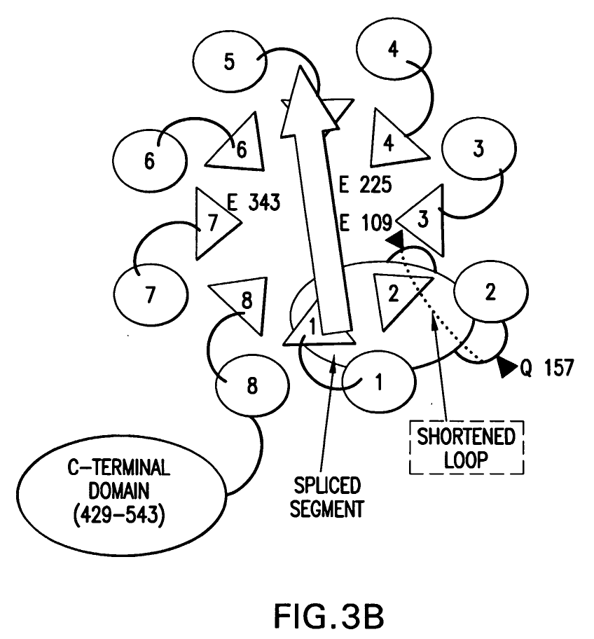

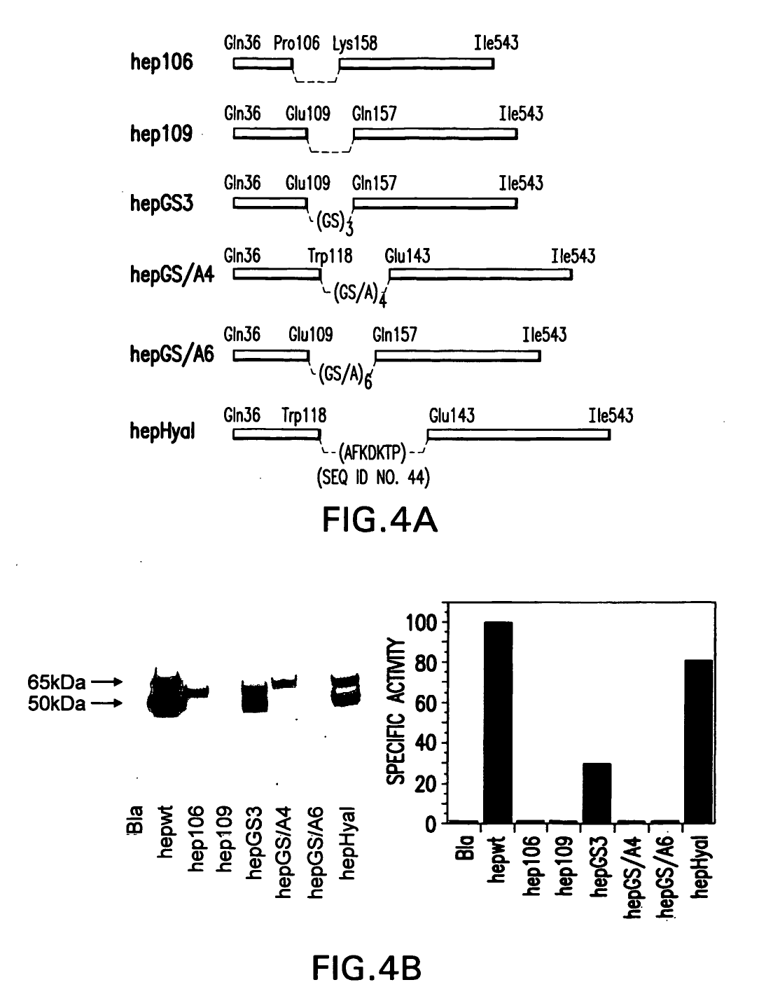

Following this hypothesis, a model of the secondary structure of heparanase, based on multiple sequence alignments (FIGS. 3A and 3B), was built to design single chain heparanase molecules having the 8 kDa and the 50 kDa subunits covalently linked together, as described herein. The present invention shows that connecting the 8 kDA and 50 kDa fragments with a linker results in constitutively active, single chain heparanase molecules that do not require proteolytic processing. In exemplary embodiments of the invention, the two fragments were connected by grafting of a loop derived from Hirudinaria manillensis hyaluronidase or with a linker comprising three glycine-serine repeats.

It is also shown herein that by engineering endoproteinase cleavage sites at about the N and C termini of the 6 kDa intervening fragment, proteolytic processing at both sites of an at least partially purified protein leads to heparanase activation in the absence of other components. In an exemplary embodiment of this aspect of the invention, tobacco etch virus protease cleavage sites are added at the N and C termini of the 6 kDa intervening fragment, resulting in active heparanase after purification or partial purification of the encoded protein and subsequent incubation with the appropriate enzyme. The present invention provides evidence of human heparanase adopting a canonical TIM barrel fold and, advantageously, provides methods for facile production of active enzyme molecules for the identification of specific inhibitors.

The engineered proteins, nucleic acid molecules, and methods of the present invention for expressing biologically active heparanase in heterologous expression systems, particularly insect cells, characteristically produce yields of 0.5-5.0 mg/l. Furthermore, these proteins are efficiently secreted into the growth medium, whereas in mammalian cells the authentic human enzyme is mainly retained inside cells or associated with the cell membranes (Vlodavsky et al, Semin. Cancer Biol. 12: 121-129 (2002)).

Accordingly, the present invention relates to synthetic nucleic acid molecules that encode an active mammalian heparanase, wherein the nucleic acid molecules are capable of expression in high yield heterologous expression systems. The synthetic heparanase molecules provided herein present a significant advance over wild-type heparanase, which are expressed at low levels in mammalian systems and improperly processed in heterologous expression systems. The synthetic molecules of the present invention can be used in inhibitor screening assays for the development of therapeutics or pharmaceuticals to inhibit and/or treat metastatic growth and/or inflammation. Said synthetic molecules are also useful in the development of therapeutics or pharmaceuticals for the treatment and/or prevention of autoimmunity.

In one aspect of the present invention, synthetic nucleic acid molecules comprising a sequence of nucleotides that encode a mammalian heparanase protein are provided, the sequence of nucleotides comprising two consensus cleavage sites recognized by an endoproteinase, the cleavage sites located between nucleotides encoding residues 100 and 168 of the heparanase protein. This aspect of the present invention provides synthetic nucleic acid molecules that can be used in methods for carrying out the proteolytic processing of the heparanase protein, similar to the biosynthesis of wild-type heparanase, resulting in a biologically active enzyme.

Also provided herein are substantially pure polypeptides encoded by the nucleic acid molecules described above.

In a preferred embodiment of the invention, the mammalian heparanase protein is human heparanase.

The two consensus cleavage sites can be introduced anywhere between residues 100 and 168 of the heparanase protein, provided that after purification or partial purification of the encoded protein and incubation with the appropriate enzyme, the resulting fragments comprise at least one fragment that is substantially similar to the wild-type 8 kDa fragment (SEQ ID NO:15) and at least one fragment that is substantially similar to the wild-type 50 kDa fragment (SEQ ID NO:16). In a preferred embodiment of the invention, the consensus cleavage sites are located before residues G110 and K158 of the human heparanase protein, resulting in a first fragment of 8 kDa, a second “intervening fragment” of 6 kDa and a third fragment of 50 kDa following purification or partial purification of the encoded protein and subsequent incubation with the appropriate enzyme.

It is understood by one of skill in the art that cleavage sites corresponding to any endoproteinase can be engineered into the heparanase molecule to obtain active, heterodimeric heparanase, including, but not limited to, cleavage sites from tobacco etch virus, 3C protease from picomavirus, thrombin, factor Xa and enterokinase. In a preferred embodiment of the invention, the cleavage sites are from tobacco etch virus.

In another aspect of the present invention, there is provided constitutively active, single-chain mammalian heparanase nucleic acid molecules comprising a portion that encodes a mammalian heparanase protein, the protein coding portion consisting essentially of a sequence of nucleotides encoding an N-terminal fragment of about 8 kDa, a linker, and a sequence of nucleotides encoding a C-terminal fragment of about 50 kDa. This aspect of the present invention provides synthetic genes encoding heparanase that are constitutively active without proteolytic processing, wherein the synthetic gene is engineered to substantially remove the 6 kDa “intervening fragment” and replace said intervening fragment with a smaller linker.

In preferred embodiments of this aspect of the present invention, the mammalian heparanase protein is a human heparanase.

Also provided herein is a purified synthetic heparanase protein encoded by the constitutively active, single-chain mammalian heparanase gene described above.

Any sequence encoding a peptide comprising from about 1 to about 67 residues can be used as a linker in this aspect of the present invention. Said linker can be synthetic or isolated from a naturally occurring source. In an exemplary embodiment of the present invention, the linker comprises a sequence of nucleotides that encodes a central loop region of the hyaluronidase protein. It is preferred that the hyaluronidase is from H. manillensis. In other embodiments, the linker comprises a sequence of nucleotides that encodes a (GlySer)₃ linker.

The present invention further relates to recombinant vectors that comprise the synthetic nucleic acid molecules disclosed throughout this specification. These vectors may be comprised of DNA or RNA. For most cloning purposes, DNA vectors are preferred. Typical vectors include plasmids, modified viruses, baculovirus, bacteriophage, cosmids, yeast artificial chromosomes, and other forms of episomal or integrated DNA that can encode a recombinant heparanase protein. It is well within the purview of the skilled artisan to determine an appropriate vector for a particular gene transfer or other use.

An expression vector containing the synthetic nucleic acid molecules disclosed throughout this specification may be used for high-level expression of mammalian heparanase in a recombinant host cell. Expression vectors may include, but are not limited to, cloning vectors, modified cloning vectors, specifically designed plasmids or viruses. Also, a variety of bacterial expression vectors may be used to express recombinant heparanase in bacterial cells if desired. In addition, a variety of fungal cell expression vectors may be used to express recombinant heparanase in fungal cells. Further, a variety of insect cell expression vectors may be used to express recombinant protein in insect cells. In a preferred embodiment of the present invention, the vector is a baculovirus vector.

The present invention also relates to host cells transformed or transfected with vectors comprising the synthetic nucleic acid molecules of the present invention. Recombinant host cells may be prokaryotic or eukaryotic, including but not limited to, bacteria such as E. coli, fungal cells such as yeast including, but not limited to, Pichia pastoris, Hansenula polymorpha and Saccharomyces cervisiae, and insect cells, including but not limited to, Drosophila and silkworm derived cell lines. Such recombinant host cells can be cultured under suitable conditions to produce high levels of mammalian heparanase or a biologically equivalent form. As defined herein, the term “host cell” is not intended to include a host cell in the body of a transgenic human being, transgenic human fetus, or transgenic human embryo.

As stated above, the synthetic molecules of the present invention provide a significant advantage over the prior art because they are capable of expression in high-yield heterologous expression systems. The heparanase proteins encoded by the synthetic molecules provided herein are correctly processed, enzymatically active, and expressed to high levels. Therefore, in preferred embodiments of the present invention, the host cell chosen is part of a high yield heterologous expression system, including, but not limited to, insect cells, bacterial cells, and yeast cells. In a particularly preferred embodiment of the present invention, the host cell is an insect cell.

The present invention also relates to recombinant vectors and recombinant host cells, both prokaryotic and eukaryotic, which contain the nucleic acid molecules disclosed throughout this specification. The synthetic nucleic acid molecules, associated vectors, and hosts of the present invention are useful in screening assays to identify inhibitors of heparanase activity, which, are useful for the treatment of cancer, inflammation and/or autoimmunity.

In another aspect of this invention, there is provided a method of expressing mammalian heparanase in non-mammalian cells comprising: (a) transforming or transfecting non-mammalian cells with a vector comprising a sequence of nucleotides that encodes a mammalian heparanase protein, the sequence of nucleotides comprising two consensus cleavage sites recognized by an endoproteinase, the cleavage sites located between nucleotides encoding residues 100 and 168 of the heparanase protein; (b) culturing the host cell under conditions which allow expression of said heparanase protein; (c) disrupting the cells and at least partially purifying the protein; and (d) exposing the at least partially purified protein to the endoproteinase, wherein the heparanase protein is cleaved at the consensus cleavage sites.

This invention also provides substantially purified protein produced by the method described above.

In a preferred embodiment of this aspect of the invention, the mammalian heparanase is human heparanase. In a further preferred embodiment, the consensus cleavage sites are located before residues G110 and K158 of human heparanase.

In another preferred embodiment, the cleavage sites are tobacco etch protein cleavage sites.

Also provided herein is a method of expressing a single chain, constitutively active mammalian heparanase in non-mammalian cells comprising: (a) transforming or transfecting non-mammalian cells with a vector comprising a synthetic mammalian heparanase gene, wherein the synthetic gene comprises a portion that encodes the heparanase protein, the protein coding portion consisting essentially of a sequence of nucleotides encoding an N-terminal fragment of about 8 kDa, a sequence of nucleotides encoding a linker and a sequence of nucleotides encoding a C-terminal fragment of about 50 kDa; and (b) culturing the host cell under conditions which allow expression of said heparanase protein.

Also provided herein is a substantially purified protein produced by the method described above. In a further embodiment of this invention, the protein is capable of binding an antibody that is specific for wild-type heparanase.

In a preferred embodiment of this aspect of the invention, the linker comprises a central loop region of the hyaluronidase protein. In another preferred embodiment, the linker comprises a (GlySer)₃ peptide.

All publications mentioned herein are incorporated by reference for the purpose of describing and disclosing methodologies and materials that might be used in connection with the present invention. Nothing herein is to be construed as an admission that the invention is not entitled to antedate such disclosure by virtue of prior invention.

Having described preferred embodiments of the invention with reference to the accompanying drawings, it is to be understood that the invention is not limited to those precise embodiments, and that various changes and modifications may be effected therein by one skilled in the art without departing from the scope or spirit of the invention as defined in the appended claims.

The following examples illustrate, but do not limit the invention.

### EXAMPLE 1

Cloning of Heparanase from a Human Placenta cDNA Library.

Human heparanase (Accession No. AF155510) was amplified from a normal human placenta cDNA library (Invitrogen Corp., Carlsbad, Calif.) by PCR using TaKaRaLa Taq polymerase (TaKaRa Bio Inc., Otsu, Shiga, Japan). Buffer conditions were those suggested by the supplier. PCR amplification of the cDNA templates consisted of one cycle of 94° C. for one minute, followed by 35 cycles of 94° C. for 30 s, 57° C. for 30 s and 68° C. for 110 seconds. The amplified fragment was gel purified, phosphorylated, and cloned either in the BamHI site of pFAST BAC1, after filling in (baculovirus expression) or into a BamHI/EcoRI-digested pcDNA3 vector (mammalian cell expression). The following primers were used for PCR amplification and simultaneous optimization of the Kozak sequence: hHEP1-24 BamHI opti 5′-C G G G A T C C G C C G C A C C A T G C T G C T G C G C T C G A A G C C T G C G-3′ (SEQ ID NO:1); and hHEP rev 1632 5′-TCA GAT GCA AGC AGC AAC TTT GGC-3′ (SEQ ID NO:2).

### EXAMPLE 2

Construction of Single Chain Heparanase Molecules

The following constructs (hepWT, hep109 (SEQ ID NO:19, corresponding protein SEQ ID NO: 20), hep106 (SEQ ID NO:18, corresponding protein SEQ ID NO:17), hepGS3 (SEQ ID NO:22, corresponding protein SEQ ID NO:21), hepGS6 (SEQ ID NO:24, corresponding protein SEQ ID NO: 26), hepGS4 (SEQ ID NO:23, corresponding protein SEQ ID NO:25) and hepHyaluro (SEQ ID NO: 28, corresponding protein SEQ ID NO: 27)), covalently linking the 8 and 50 kDa subunits in a direct fashion (hep109 and hep106), linking the subunits via glycine-serine spacers (hepGS3, hepGS4 and hepGS6) or by grafting a loop region from the enzyme hyaluronidase (Hyaluro) were generated by standard PCR mutagenesis using the indicated primers:

hHEP1-24 BamHI opti (SEQ ID NO:1) and hHEP rev 1632 (SEQ ID NO:2)
hep109M1 E109-Q157 1543
Mutagenic primer: hHEP 304/504
5′- C T A A T T T T C G A T C C C A A G A A G G A A A A A A A G T T C A A
G A A C A G C A C C T A C -3′
(SEQ ID NO:3)
hep106M1 P106-K158 1543
Mutagenic primer: hHEP 291/504 bis
5′- A A G A C A G A C T T C C T A A T T T T C G A T C C C A A A A A G T T
C A A G A A C A G C A C C T A C-3′
(SEQ ID NO:4)
bepGS3 M1 E109-(GS)₃-Q157 1543
Mutagenic primer: hHEP 304(GS3)504
5′- C T A A T T T T C G A T C C C A A G A A G G A A G G T A G C G G T T C
C G G C T C T A A A A A G T T C A A G A A C-3′
(SEQ ID NO:5)
bepGS6 M1 E109-(GS)₆-Q157 1543
Mutagenic primer: hHEP 304(GS6 Ala)
5′- C T A A T T T T C G A T C C C A A G A A G G A A G G T A G C G G C G C
T G G A T C A G G G G C A G C A G G A T C C G G C G C C A A A A A G T T C
A A G A A C A G C A C C T A C
(SEQ ID NO:6)
hepGS4 M1 W118-(GS)₄-E143 1543
Mutagenic primer:hHEP 329(GS4 Ala)
5′- A C C T T T G A A G A G A G A A G T T A C T G G G G T T C A G G G G C
A G G A T C C G G C G C C G A A T G G C C C T A C C A G G A G C A A T T G
(SEQ ID NO:7)
hepHyaluro M1 W118-(AFKDKPT) (SEQ ID NO:8)-E143 1543
Mutagenic primer: hHEP Hyaluro
5′-:A C C T T T G A A G A G A G A A G T T A C T G G G C C T T C A A G G A
C A A G A C C C C C G A A T G G C C C T A C C A G G A G C A A T T G -3′
(SEQ ID NO:9)

### EXAMPLE 3

Construction of Heparanase Molecules with Engineered Protease Cleavage Sites

To construct an engineered heparanase molecule inserting the consensus cleavage site for the tobacco etch virus (TEV) protease flanked by GS repeats (E109-GSGSENLYFQ-GSG-G110 (SEQ ID NO:10), the scissile bond being located between Q and G) between amino acids E109 and G110, PCR mutagenesis was employed using wt heparanase as a template and the primers hHEP1-24 BamHI opti (SEQ ID NO:1) and hHEP rev 1632 (SEQ ID NO:2) and the mutagenic primer TEV110 bis 5′-G G C A G C G G A T C T G A G A A C C T G T A C T T C C A G G G T T C C G G T T C A A C C T T T G A A G A G A G A A G T T A C-3′(SEQ ID NO:1).

To construct an engineered heparanase having TEV-cleavage sites both between residues E109/G110 and Q157/K158 the TEV110 construct (SEQ ID NO:30; corresponding protein SEQ ID NO:31) was used as a template to insert the sequence Q157-GSGSENLYFQ-GSGS-K158 (SEQ ID NO:12) by PCR mutagenesis using the mutagenic primer TEV158 ter 5′-T C T G G A T C C G G T G A A A A T C T C T A T T T T C A G G G C T C A G G A A G T A A A A A G T T C A A G A A C A G C A C C T A C-3′ (SEQ ID NO:13), to produce hepTEV110/158 (SEQ ID NOs: 29 and 32).

All constructs were sequenced on both strands to assure that no mutations were introduced by PCR and cloned into pFASTBAC1 as described above.

### EXAMPLE 4

Transient Expression of Heparanase Molecules in COS7 Cells.

Cells were grown in Dulbecco's MEM (Gibco BRL, Gaithersburg, Md.). All constructs were cloned into the eukaryotic expression plasmid pcDNA3 (Invitrogen). A vector encoding the reporter gene β-lactamase (BLA) was co-transfected in order to check transfection efficiency of each construct. The quantity of each transfected vector was adjusted in order to obtain comparable transfection efficiencies. Transient transfection of COS7 cells was obtained using the fuGENE 6 Transfection Reagent (Roche, Basel, Switzerland) according to manufacturer's instructions. 24 hours after transfection, efficiency was assessed by fluorimetric detection of BLA-positive cells. 96 hours after transfection, cells were harvested and resuspended in lysis buffer (50 mM Tris-HCl pH 7.5, 150 mM NaCl, 0.5% Triton) containing Complete protease inhibitor cocktail (Roche). The lysis was carried out on ice for 30 minutes. After centrifugation at 14000 rpm for 30 minutes, the heparanase containing supernatants were recovered and partially purified as outlined below.

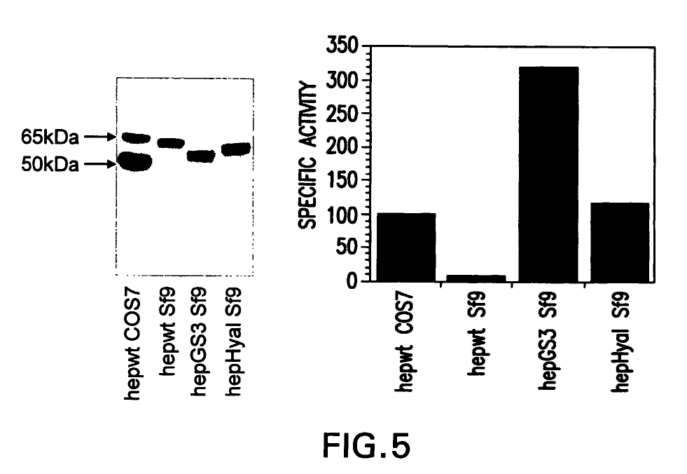

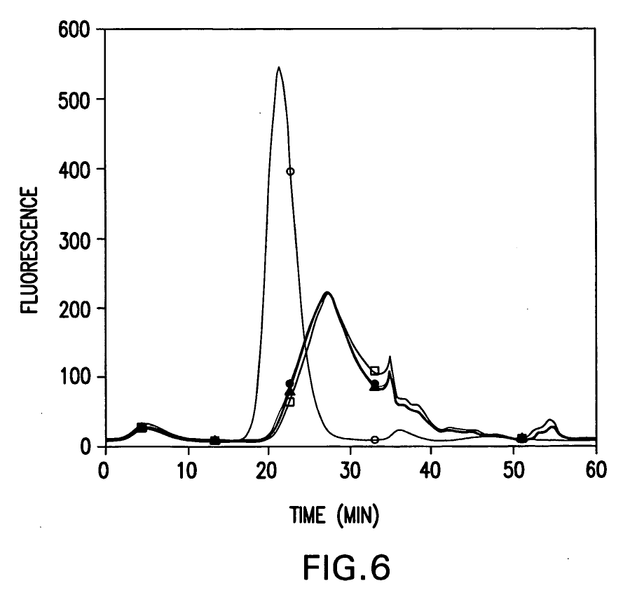

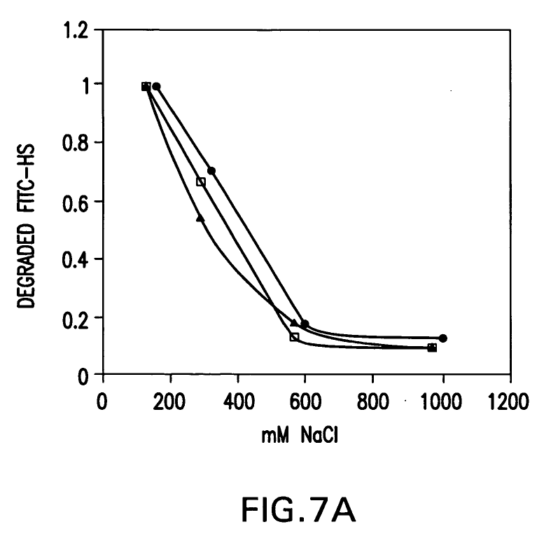

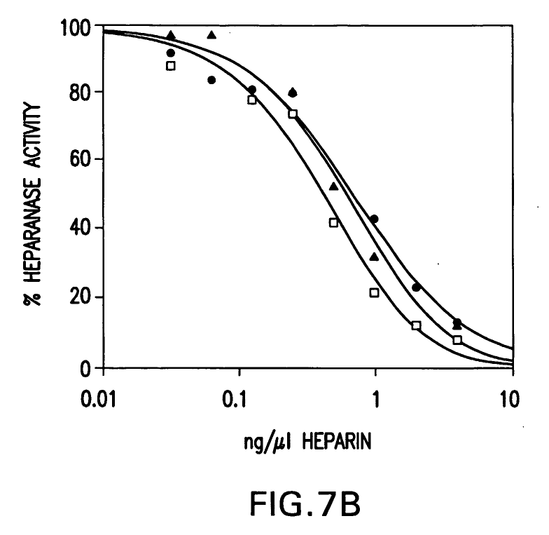

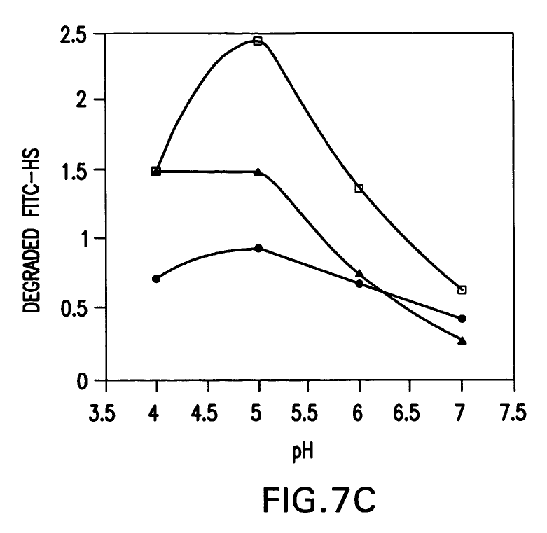

Heparanase constructs were expressed in COS-7 cells, which are devoid of endogenous heparanse activity, by transient transfection. Heparanase was extracted from cell lysates by heparin affinity chromatography and quantified on Western blots. In parallel, heparanase enzymatic activity was determined with either the radiometric or fluorimetric assay (FIG. 5). From Western blot analysis, we concluded that wt heparanase as well as the single chain constructs GS3 and hyaluro are efficiently expressed and processed, whereas constructs 106 and GS4 are expressed but not processed. Expression levels of constructs 109 and GS6 were extremely low and barely detectable by Western blot analysis. Only the wt, GS3 and hyaluro constructs showed enzymatic activity. We conclude that single chain constructs 106 and GS4 are inactive whereas constructs 109 and GS6 are probably unstable. Since GS3 and hyaluro are active but are processed despite the changes that were introduced in the cleavage sites we can not draw any conclusion with respect to the intrinsic activity of the precursors. We therefore proceeded with the expression in cells that are devoid of the enzyme(s) responsible for heparanase processing.

### EXAMPLE 5

Expression of Heparanase Molecules in Insect Cells.

Recombinant baculoviruses containing the heparanase constructs were generated using the Bac to Bac expression system (Invitrogen). Recombinant baculoviruses were used to infect Sf9 insect cells (50×10⁶ cells per T-175 flask) grown in Grace's insect medium with 10% FBS. Cells were collected 48 h after infection, and centrifuged at 500 g for 5 minutes. Cell lysates were prepared as above, except the lysis buffer contained 500 mM NaCl instead of the 150 mM used for COS7, which improved protein quantity in the soluble fraction.

The three heparanase constructs that showed enzymatic activity when produced in COS-7 cells were transferred into a baculovirus expression system. The proteins were expressed in Sf9 cells and purified by heparin affinity chromatography. Western blot analysis showed that, in contrast to what was observed in COS-7 cells, no processing of wt or mutant heparanases occurred in this expression system. Analysis of the enzymatic activity of the purified single chain proteins by the fluorimetric activity assay revealed that the unprocessed wt enzyme had a very low activity, whereas the unprocessed GS3 and hyaluro proteins resulted to be highly active, with specific activities comparable to those observed with the correctly processed wild type enzyme produced in COS-7 cells.

GS3 and hyaluro where undistinguishable from the wild type recombinant enzyme extracted from COS-7 cells or from the authentic wt enzyme partially purified from HCT-116 cells on what concerns pH and ionic strength dependence of the enzymatic activity and were inhibited with similar potencies by heparin.

The constructs having TEV cleavage sites at positions 109/110 and 109/110+157/158 were expressed, purified on a heparin affinity column and digested overnight at room temperature with TEV protease (0.5 μM) in 50 mM Mes pH 6.0, 10% glycerol, 0.5 mM EDTA. Complete processing was observed in both cases, however only the double mutant, carrying TEV sequences at both cleavage junctions was activated by this treatment, indicating that processing at the E109/G110 junction only is not sufficient for eliciting activation of heparanase.

### EXAMPLE 6

Purification of Recombinant Heparanase Constructs by Heparin Sepharose Affinity Chromatography.

Cell lysates from COS7 or Sf9 insect cells were passed through 500 μl Heparin Sepharose CL-6B (Amersham, Piscataway, N.J.) by gravity. The column was washed with 2 ml of lysis buffer, then with 2 ml of 50 mM Tris-HCl pH 7.5, 500 mM NaCl, and heparanase was eluted with 2 ml of 50 mM Tris-HCl pH 7.5, 1 M NaCl and concentrated about 5 fold with a Biomax-30K centrifugal concentrator (Millipore, Bedford, Mass.). 10% glycerol was added and the protein was stored in aliquots at −80° C. Protein concentration was determined using the BIO-RAD Protein Assay.

### EXAMPLE 7

Large Scale Expression and Purification

Sf21 (or Sf9) cells were adapted to growth in serum free medium (Sf-900 II SFM, Invitrogen). Cells were infected with recombinant baculoviruses encoding heparanase constructs at multiplicities of infection varying between 1-10. 3 l of infected cells were grown in spinner flasks at 27° C. under a constant flux of sterile air. 48-96 hours after the infection cells were collected and separated from the medium by centrifugation. Synthetic and wt heparanase were found in both the cell pellet and in the supernatant. To extract synthetic heparanase from the cell pellet, cells were disrupted as outlined above. Cell lysates or the crude medium supernatant were filtered on a 0.22μ filter and loaded on a 20 ml-HyperD Heparin column (Biosepra Inc., Marlboro, Mass.) equilibrated with 50 mM Tris-HCl pH 7.5, 150 mM NaCl. Synthetic or wt heparanase were eluted by applying a linear 0.15-1M NaCl gradient in 50 mM Tris HCl pH 7.5. Recombinant proteins eluted at NaCl concentrations>500 mM. The pooled, heparanase-containing Heparin-column fractions were dialyzed overnight against 50 mM HEPES pH 7.5 and loaded on a Source S column (Amersham) equilibrated in the same buffer. Heparanase constructs eluted with 400-600 mM NaCl. Proteins were purified to homogeneity by a further chromatographic step on a 15/30 Superdex 75 size exclusion column. The purified proteins were aliquoted, shock-frozen in liquid nitrogen and stored at −80° C.

### EXAMPLE 8

Western Blotting.

Rabbit polyclonal antibodies were generated against a peptide contained within the 50 kDa subunit (EPNSFLKKADIFINGSQ (SEQ ID NO:14), corresponding to amino acids 225 to 241 and containing the additional sequence GGC at its C-terminus). Antisera were immunopurified using the immunogen peptide immobilized on a thiopropyl Sepharose resin (Amersham). 10 μl of proteins eluted from the heparin column were subjected to 10% SDS-polyacrylamide gel electrophoresis and transferred onto Protran BA 83 Cellulosenitrate membrane (Schleicher & Schuell Bioscience, Keene, N.H.). After saturation of non specific binding with 5% milk, the membrane was incubated with the polyclonal antibody described above diluted 1:500 in 5% milk, TBS and 0.05% Tween20 over night at 4° C. After washing, the membrane was incubated with anti-rabbit horseradish peroxidase-conjugated antibody diluted 1:5000 for 30′ at room temperature. The immunoreactive bands were detected by SuperSignal West Pico Chemiluminescent Substrate (Pierce Biotechnology, Rockford, Ill.). Finally the membrane was exposed to BIOMAX MR film (Kodak) for 10 s.

### EXAMPLE 9

Fluorometric Labeling of Heparan Sulfate

Heparan sulfate sodium salt from bovine kidney (Sigma-Aldrich Corp., St. Louis, Mo.) was labeled with fluorescein isothiocyanate (FITC) as previously described (Toyoshima and Nakajima, J. Biol. Chem. 274: 24153-24160 (1999)). 5 mg of heparan sulfate and 5 mg of FITC were dissolved in 1 ml of 0.1 M Na₂CO₃ pH 9.5 and incubated over night at 4° C. in the dark. The solution was then loaded on MicroSpin G-25 columns in order to separate FITC labeled Heparan Sulfate (FITC-HS) from unreacted FITC. The FITC-HS was subjected to a first gel-filtration chromatographic step through Sephacryl S-300 in 150 mM NaCl, 25 mM Tris-HCl pH=7.5 buffer to separate the high molecular weight heparan sulfate species. The colored fractions were pooled, concentrated with Biomax-10K centrifugal concentrator (Millipore) and rechromatographed on Sephacryl S-300 (as above) in order to obtain heparan sulfate species with homogeneous molecular weight. The eluted fractions were analyzed by HPLC Superdex 75™ (Pharmacia Biotech) chromatography system. The fluorescence in each fraction was measured by an L-7485 fluorescence detector (Merck Hitachi). We obtained four main fractions with different molecular weight heparan sulfate products. The quantity of FITC-HS in each fraction was measured with the Blyscan Glycosaminoglycan Assay (Biocolor Ltd., Belfast, Northern Ireland).

### EXAMPLE 10

Fluorimetric Assay.

This assay is based on the degradation of FITC-HS monitored by HPLC size exclusion chromatography. 8 μl of purified heparanase was incubated with 5 μL of FITC-HS in a 50 μl of 50 mM MES pH 6, 10% glycerol (heparanase activity buffer, HAB). The reaction mixture was incubated at room temperature for a defined period and the reaction was stopped by the addition of 50 μg of heparin. The mixture was then filtered using Ultrafree-MC centrifugal filter Devices (Millipore). 20 μl were injected on a Superdex 75™ (Pharmacia Biotech) column equilibrated in buffer 50 mM Hepes pH 7.5 150 mM Na₂SO₄ and connected to a Merck-Hitachi HPLC system. Fluorescent heparan sulfate degradation products were detected by an L-7485 fluorescence detector. Heparanase activity was assessed by monitoring the increase in lower molecular weight heparan sulfate species compared with the intact FITC-HS and quantified by peak area integration.

### EXAMPLE 11

Radiometric Labeling and Biotinylation at the Reducing End of Heparan Sulfate

10 mg of heparan sulfate sodium salt from bovine kidney (Sigma) were partially N-de-acetylated and re-acetylated with [³H] acetic anhydride as previously described (Freeman and Parish, Biochem. J. 325: 229-237 (1997)). Tritiated heparan sulfate was then subjected to reductive amination at the reducing end as described. Tritiated, reductively aminated heparan sulfate was further conjugated to biotin using EZ-Link Sulfo-NHS-LC-Biotin (Pierce). This biotin analog has an N-hydroxysuccinimido ester moiety that can react with the amino group generated at the reducing end of the heparan sulfate molecules. We calculated a recovery in about 5 mg of tritiated heparan sulfate, reductively aminated and resuspended in 1 ml of H₂O (an estimated final concentration of 100 μM taking into account an average in heparan sulfate molecular weight of 500 KDa). To 100 μl of this solution 1 mg of EZ-Link Sulfo-NHS-LC-Biotin (about 100-fold molar excess) and 20 μL of phosphate buffer pH 7.5 were added. The reaction mixture was incubated overnight at room temperature. The reaction mixture was then loaded on PD-10 desalting column in order to separate biotinylated, tritiated heparan sulfate from unreacted biotin. We finally obtained four fractions (1 ml each), which were tested for their ability to be immobilized on Reacti-Bind Streptavidin High Binding Capacity Coated Plates (Pierce).

### EXAMPLE 12

Radiometric Assay.

This assay is based on the degradation of tritiated heparan sulfate immobilized on microplate. Each well of the Reacti-Bind Streptavidin High Binding Capacity Coated Plates was pre-treated according to manufacturer's instructions. Initially, different amounts of each fraction of tritiated, biotinylated heparan sulfate obtained after PD-10 desalting column were added to each well (in duplicate) in PBS to a final volume of 100 μl. After assesseing that the maximum binding is obtained with a volume of fraction 2 corresponding to 100×10³ d.p.m. this amount was always used. The binding was carried out over night at room temperature. The wells were then washed three times with PBS and twice with HAB. 10 μl of purified heparanase were added to each well in HAB to a final volume of 100 μl. The wells were incubated at room temperature for 2-24 hours. Finally, the liberated radioactivity due to tritiated heparan sulfate products generated by heparanase in each well was measured and normalized against a buffer blank.

### EXAMPLE 13

Determination of Specific Activity of Heparanase Constructs.

Specific activities of the heparanase constructs either transiently expressed in COS7 cells or expressed in the baculovirus system were determined as follows:

$$Specific activity = \frac{normalized activity ( d . p . m \text{/} μ l )}{normalized densitometric volume ( volume \text{/} μ l )}$$

In detail, activity of partially purified heparanse constructs was determined in the radiometric assay by titrating each preparation in such a way that a linear dose-activity relationship was observed. These titrations were repeated three times with each preparation and a mean, normalized activity (d.p.m./μl) was calculated. Protein expression was determined by the Western blotting experiments: the chemiluminescent readout was quantified by densitometry. Again, experiments were repeated three times and mean values were determined. The specific activity was obtained by dividing the normalized activity (d.p.m./μl) by the normalized densitometric volume (volume/μl).

1. A synthetic nucleic acid molecule comprising a sequence of nucleotides that encodes a mammalian heparanase protein, the sequence of nucleotides comprising two consensus cleavage sites recognized by an endoproteinase, the cleavage sites located between nucleotides encoding residues 100 and 168 of the heparanase protein.

2. A vector comprising the nucleic acid molecule of claim 1.

3. (canceled)

4. A host cell comprising the vector of claim 3.

5. The host cell of claim 4, wherein the host cell is an insect cell or a yeast cell.

6. (canceled)

7. The host cell of claim 5, wherein the host cell is a yeast cell which is selected from the group consisting of: Pichia pastoris, Hansenula polymorpha and Saccharomyces cervisiae.

8. The synthetic nucleic acid molecule of claim 1, wherein the heparanase protein is human heparanase.

9. The synthetic nucleic acid molecule of claim 8, wherein the consensus cleavage sites are located before residues G110 and K158 of the human heparanase protein.

10. The synthetic nucleic acid molecule of claim 8, wherein the consensus cleavage sites are selected from the group consisting of: tobacco etch virus (TEV) protease cleavage sites, 3C protease cleavage sites from picornavirus, thrombin protease cleavage sites, enterokinase cleavage sites and factor Xa cleavage sites.

11. A synthetic mammalian heparanase nucleic acid molecule comprising a portion that encodes a mammalian heparanase protein, the protein coding portion consisting essentially of a sequence of nucleotides encoding an N-terminal fragment of about 8 kDa, a linker, and a sequence of nucleotides encoding a C-terminal fragment of about 50 kDa, wherein the N-terminal and C-terminal fragments encode protein fragments that are substantially similar to wild-type heparanase fragments, and wherein the encoded heparanase protein is constitutively active.

12. The synthetic nucleic acid molecule of claim 11, wherein the protein coding portion encodes human heparanase.

13. The synthetic nucleic acid molecule of claim 11, wherein the linker comprises a sequence of nucleotides that encodes a central loop region of the hyaluronidase protein.

14. The synthetic nucleic acid molecule of claim 13, wherein the hyaluronidase is from H. manillensis.

15. The synthetic nucleic acid molecule of claim 12, wherein the linker comprises a sequence of nucleotides that encodes a (GlySer)₃ linker.

16. A vector comprising the synthetic nucleic acid molecule of claim 12.

17. A host cell comprising the vector of claim 16.

18. The host cell of claim 17 which is an insect cell or a yeast cell.

19. A purified synthetic heparanase protein encoded by the gene of claim 12.

20. A method of expressing mammalian heparanase in non-mammalian cells comprising:
(a) transforming or transfecting non-mammalian cells with a vector comprising a sequence of nucleotides that encodes a mammalian heparanase protein, the sequence of nucleotides comprising two consensus cleavage sites recognized by an endoproteinase, the cleavage sites located between residues 100 and 168 of the heparanase protein;
(b) culturing the host cell under conditions which allow expression of said heparanase protein;
(c) disrupting the cells and at least partially purifying the heparanase protein; and
(d) exposing the at least partially purified heparanase protein to the endoproteinase, wherein the heparanase protein is cleaved at the consensus cleavage sites.

21. (canceled)

22. A method of expressing a single chain, constitutively active mammalian heparanase in non-mammalian cells comprising:
(a) transforming or transfecting non-mammalian cells with a vector comprising a synthetic mammalian heparanase gene, wherein the synthetic gene comprises a portion that encodes the heparanase protein, the protein coding portion consisting essentially of a sequence of nucleotides encoding an N-terminal fragment of about 8 kDa, a sequence of nucleotides encoding a linker and a sequence of nucleotides encoding a C-terminal fragment of about 50 kDa, wherein the N-terminal and C-terminal fragments encode protein fragments that are substantially similar to wild-type fragments; and
(b) culturing the host cell under conditions which allow expression of said heparanase protein

23. (canceled)

24. The method of claim 22 wherein the linker comprises a central (GlySer)₃.

25. (canceled)


SEQUENCE LISTING (44 sequences)

SEQ ID NO: 1 - DNA, 40 bp, Artificial Sequence
  PCR Primer
cgggatccgccgcaccatgctgctgcgctcgaagcctgcg

SEQ ID NO: 2 - DNA, 24 bp, Artificial Sequence
  PCR Primer
tcagatgcaagcagcaactttggc

SEQ ID NO: 3 - DNA, 48 bp, Artificial Sequence
  PCR Primer
ctaattttcgatcccaagaaggaaaaaaagttcaagaacagcacctac

SEQ ID NO: 4 - DNA, 51 bp, Artificial Sequence
  PCR Primer
aagacagacttcctaattttcgatcccaaaaagttcaagaacagcacctac

SEQ ID NO: 5 - DNA, 57 bp, Artificial Sequence
  PCR Primer
ctaattttcgatcccaagaaggaaggtagcggttccggctctaaaaagttcaagaac

SEQ ID NO: 6 - DNA, 87 bp, Artificial Sequence
  PCR Primer
ctaattttcgatcccaagaaggaaggtagcggcgctggatcaggggcagcaggatccggcgccaaaaagttcaagaacagcacctac

SEQ ID NO: 7 - DNA, 72 bp, Artificial Sequence
  PCR Primer
acctttgaagagagaagttactggggttcaggggcaggatccggcgccgaatggccctaccaggagcaattg

SEQ ID NO: 8 - protein, 8 aa, Artificial Sequence
  Peptide
WAFKDKPT

SEQ ID NO: 9 - DNA, 69 bp, Artificial Sequence
  PCR Primer
acctttgaagagagaagttactgggccttcaaggacaagacccccgaatggccctaccaggagcaattg

SEQ ID NO: 10 - protein, 15 aa, Artificial Sequence
  Peptide
EGSGSENLYFQGSGG

SEQ ID NO: 11 - DNA, 63 bp, Artificial Sequence
  PCR Primer
ggcagcggatctgagaacctgtacttccagggttccggttcaacctttgaagagagaagttac

SEQ ID NO: 12 - protein, 16 aa, Artificial Sequence
  Peptide
QGSGSENLYFQGSGSK

SEQ ID NO: 13 - DNA, 66 bp, Artificial Sequence
  PCR Primer
tctggatccggtgaaaatctctattttcagggctcaggaagtaaaaagttcaagaacagcacctac

SEQ ID NO: 14 - protein, 17 aa, Artificial Sequence
  Peptide
EPNSFLKKADIFINGSQ

SEQ ID NO: 15 - protein, 74 aa, Human
QDVVDLDFFTQEPLHLVSPSFLSVTIDANLATDPRFLILLGSPKLRTLARGLSPAYLRFGGTKTDFLIFDPKKE

SEQ ID NO: 16 - protein, 386 aa, Human
KKFKNSTYSRSSVDVLYTFANCSGLDLIFGLNALLRTADLQWNSSNAQLLLDYCSSKGYNISWELGNEPNSFLKKADIFINGSQLGEDFIQLHKLLRKSTFKNAKLYGPDVGQPRRKTAKMLKSFLKAGGEVIDSVTWHHYYLNGRTATREDFLNPDVLDIFISSVQKVFQVVESTRPGKKVWLGETSSAYGGGAPLLSDTFAAGFMWLDKLGLSARMGIEVVMRQVFFGAGNYHLVDENFDPLPDYWLSLLFKKLVGTKVLMASVQGSKRRKLRVYLHCTNTDNPRYKEGDLTLYAINLHNVTKYLRLPYPFSNKQVDKYLLRPLGPHGLLSKSVQLNGLTLKMVDDQTLPPLMEKPLRPGSSLGLPAFSYSFFVIRNAKVAACI

SEQ ID NO: 17 - protein, 492 aa, Artificial Sequence
  hep 106
MLLRSKPALPPPLMLLLLGPLGPLSPGALPRPAQAQDVVDLDFFTQEPLHLVSPSFLSVTIDANLATDPRFLILLGSPKLRTLARGLSPAYLRFGGTKTDFLIFDPKKFKNSTYSRSSVDVLYTFANCSGLDLIFGLNALLRTADLQWNSSNAQLLLDYCSSKGYNISWELGNEPNSFLKKADIFINGSQLGEDFIQLHKLLRKSTFKNAKLYGPDVGQPRRKTAKMLKSFLKAGGEVIDSVTWHHYYLNGRTATREDFLNPDVLDIFISSVQKVFQVVESTRPGKKVWLGETSSAYGGGAPLLSDTFAAGFMWLDKLGLSARMGIEVVMRQVFFGAGNYHLVDENFDPLPDYWLSLLFKKLVGTKVLMASVQGSKRRKLRVYLHCTNTDNPRYKEGDLTLYAINLHNVTKYLRLPYPFSNKQVDKYLLRPLGPHGLLSKSVQLNGLTLKMVDDQTLPPLMEKPLRPGSSLGLPAFSYSFFVIRNAKVAACI

SEQ ID NO: 18 - DNA, 1479 bp, Artificial Sequence
  hep 106
atgctgctgcgctcgaagcctgcgctgccgccgccgctgatgctgctgctcctggggccgctgggtcccctctcccctggcgccctgccccgacctgcgcaagcacaggacgtcgtggacctggacttcttcacccaggagccgctgcacctggtgagcccctcgttcctgtccgtcaccattgacgccaacctggccacggacccgcggttcctcatcctcctgggttctccaaagcttcgtaccttggccagaggcttgtctcctgcgtacctgaggtttggtggcaccaagacagacttcctaattttcgatcccaaaaagttcaagaacagcacctactcaagaagctctgtagatgtgctatacacttttgcaaactgctcaggactggacttgatctttggcctaaatgcgttattaagaacagcagatttgcagtggaacagttctaatgctcagttgctcctggactactgctcttccaaggggtataacatttcttgggaactaggcaatgaacctaacagtttccttaagaaggctgatattttcatcaatgggtcgcagttaggagaagattttattcaattgcataaacttctaagaaagtccaccttcaaaaatgcaaaactctatggtcctgatgttggtcagcctcgaagaaagacggctaagatgctgaagagcttcctgaaggctggtggagaagtgattgattcagttacatggcatcactactatttgaatggacggactgctaccagggaagattttctaaaccctgatgtattggacatttttatttcatctgtgcaaaaagttttccaggtggttgagagcaccaggcctggcaagaaggtctggttaggagaaacaagctctgcatatggaggcggagcgcccttgctatccgacacctttgcagctggctttatgtggctggataaattgggcctgtcagcccgaatgggaatagaagtggtgatgaggcaagtattctttggagcaggaaactaccatttagtggatgaaaacttcgatcctttacctgattattggctatctcttctgttcaagaaattggtgggcaccaaggtgttaatggcaagcgtgcaaggttcaaagagaaggaagcttcgagtataccttcattgcacaaacactgacaatccaaggtataaagaaggagatttaactctgtatgccataaacctccataatgtcaccaagtacttgcggttaccctatcctttttctaacaagcaagtggataaataccttctaagacctttgggacctcatggattactttccaaatctgtccaactcaatggtctaactctaaagatggtggatgatcaaaccttgccacctttaatggaaaaacctctccggccaggaagttcactgggcttgccagctttctcatatagtttttttgtgataagaaatgccaaagttgctgcttgcatctga

SEQ ID NO: 19 - DNA, 1488 bp, Artificial Sequence
  hep 109
atgctgctgcgctcgaagcctgcgctgccgccgccgctgatgctgctgctcctggggccgctgggtcccctctcccctggcgccctgccccgacctgcgcaagcacaggacgtcgtggacctggacttcttcacccaggagccgctgcacctggtgagcccctcgttcctgtccgtcaccattgacgccaacctggccacggacccgcggttcctcatcctcctgggttctccaaagcttcgtaccttggccagaggcttgtctcctgcgtacctgaggtttggtggcaccaagacagacttcctaattttcgatcccaagaaggaaaaaaagttcaagaacagcacctactcaagaagctctgtagatgtgctatacacttttgcaaactgctcaggactggacttgatctttggcctaaatgcgttattaagaacagcagatttgcagtggaacagttctaatgctcagttgctcctggactactgctcttccaaggggtataacatttcttgggaactaggcaatgaacctaacagtttccttaagaaggctgatattttcatcaatgggtcgcagttaggagaagattttattcaattgcataaacttctaagaaagtccaccttcaaaaatgcaaaactctatggtcctgatgttggtcagcctcgaagaaagacggctaagatgctgaagagcttcctgaaggctggtggagaagtgattgattcagttacatggcatcactactatttgaatggacggactgctaccagggaagattttctaaaccctgatgtattggacatttttatttcatctgtgcaaaaagttttccaggtggttgagagcaccaggcctggcaagaaggtctggttaggagaaacaagctctgcatatggaggcggagcgcccttgctatccgacacctttgcagctggctttatgtggctggataaattgggcctgtcagcccgaatgggaatagaagtggtgatgaggcaagtattctttggagcaggaaactaccatttagtggatgaaaacttcgatcctttacctgattattggctatctcttctgttcaagaaattggtgggcaccaaggtgttaatggcaagcgtgcaaggttcaaagagaaggaagcttcgagtataccttcattgcacaaacactgacaatccaaggtataaagaaggagatttaactctgtatgccataaacctccataatgtcaccaagtacttgcggttaccctatcctttttctaacaagcaagtggataaataccttctaagacctttgggacctcatggattactttccaaatctgtccaactcaatggtctaactctaaagatggtggatgatcaaaccttgccacctttaatggaaaaacctctccggccaggaagttcactgggcttgccagctttctcatatagtttttttgtgataagaaatgccaaagttgctgcttgcatctga

SEQ ID NO: 20 - protein, 495 aa, Artificial Sequence
  hep 109
MLLRSKPALPPPLMLLLLGPLGPLSPGALPRPAQAQDVVDLDFFTQEPLHLVSPSFLSVTIDANLATDPRFLILLGSPKLRTLARGLSPAYLRFGGTKTDFLIFDPKKEKKFKNSTYSRSSVDVLYTFANCSGLDLIFGLNALLRTADLQWNSSNAQLLLDYCSSKGYNISWELGNEPNSFLKKADIFINGSQLGEDFIQLHKLLRKSTFKNAKLYGPDVGQPRRKTAKMLKSFLKAGGEVIDSVTWHHYYLNGRTATREDFLNPDVLDIFISSVQKVFQVVESTRPGKKVWLGETSSAYGGGAPLLSDTFAAGFMWLDKLGLSARMGIEVVMRQVFFGAGNYHLVDENFDPLPDYWLSLLFKKLVGTKVLMASVQGSKRRKLRVYLHCTNTDNPRYKEGDLTLYAINLHNVTKYLRLPYPFSNKQVDKYLLRPLGPHGLLSKSVQLNGLTLKMVDDQTLPPLMEKPLRPGSSLGLPAFSYSFFVIRNAKVAACI

SEQ ID NO: 21 - protein, 501 aa, Artificial Sequence
  hep GS3
MLLRSKPALPPPLMLLLLGPLGPLSPGALPRPAQAQDVVDLDFFTQEPLHLVSPSFLSVTIDANLATDPRFLILLGSPKLRTLARGLSPAYLRFGGTKTDFLIFDPKKEGSGSGSKKFKNSTYSRSSVDVLYTFANCSGLDLIFGLNALLRTADLQWNSSNAQLLLDYCSSKGYNISWELGNEPNSFLKKADIFINGSQLGEDFIQLHKLLRKSTFKNAKLYGPDVGQPRRKTAKMLKSFLKAGGEVIDSVTWHHYYLNGRTATREDFLNPDVLDIFISSVQKVFQVVESTRPGKKVWLGETSSAYGGGAPLLSDTFAAGFMWLDKLGLSARMGIEVVMRQVFFGAGNYHLVDENFDPLPDYWLSLLFKKLVGTKVLMASVQGSKRRKLRVYLHCTNTDNPRYKEGDLTLYAINLHNVTKYLRLPYPFSNKQVDKYLLRPLGPHGLLSKSVQLNGLTLKMVDDQTLPPLMEKPLRPGSSLGLPAFSYSFFVIRNAKVAACI

SEQ ID NO: 22 - DNA, 1506 bp, Artificial Sequence
  hep GS3
atgctgctgcgctcgaagcctgcgctgccgccgccgctgatgctgctgctcctggggccgctgggtcccctctcccctggcgccctgccccgacctgcgcaagcacaggacgtcgtggacctggacttcttcacccaggagccgctgcacctggtgagcccctcgttcctgtccgtcaccattgacgccaacctggccacggacccgcggttcctcatcctcctgggttctccaaagcttcgtaccttggccagaggcttgtctcctgcgtacctgaggtttggtggcaccaagacagacttcctaattttcgatcccaagaaggaaggtagcggttccggctctaaaaagttcaagaacagcacctactcaagaagctctgtagatgtgctatacacttttgcaaactgctcaggactggacttgatctttggcctaaatgcgttattaagaacagcagatttgcagtggaacagttctaatgctcagttgctcctggactactgctcttccaaggggtataacatttcttgggaactaggcaatgaacctaacagtttccttaagaaggctgatattttcatcaatgggtcgcagttaggagaagattttattcaattgcataaacttctaagaaagtccaccttcaaaaatgcaaaactctatggtcctgatgttggtcagcctcgaagaaagacggctaagatgctgaagagcttcctgaaggctggtggagaagtgattgattcagttacatggcatcactactatttgaatggacggactgctaccagggaagattttctaaaccctgatgtattggacatttttatttcatctgtgcaaaaagttttccaggtggttgagagcaccaggcctggcaagaaggtctggttaggagaaacaagctctgcatatggaggcggagcgcccttgctatccgacacctttgcagctggctttatgtggctggataaattgggcctgtcagcccgaatgggaatagaagtggtgatgaggcaagtattctttggagcaggaaactaccatttagtggatgaaaacttcgatcctttacctgattattggctatctcttctgttcaagaaattggtgggcaccaaggtgttaatggcaagcgtgcaaggttcaaagagaaggaagcttcgagtataccttcattgcacaaacactgacaatccaaggtataaagaaggagatttaactctgtatgccataaacctccataatgtcaccaagtacttgcggttaccctatcctttttctaacaagcaagtggataaataccttctaagacctttgggacctcatggattactttccaaatctgtccaactcaatggtctaactctaaagatggtggatgatcaaaccttgccacctttaatggaaaaacctctccggccaggaagttcactgggcttgccagctttctcatatagtttttttgtgataagaaatgccaaagttgctgcttgcatctga

SEQ ID NO: 23 - DNA, 1584 bp, Artificial Sequence
  hep GS4
atgctgctgcgctcgaagcctgcgctgccgccgccgctgatgctgctgctcctggggccgctgggtcccctctcccctggcgccctgccccgacctgcgcaagcacaggacgtcgtggacctggacttcttcacccaggagccgctgcacctggtgagcccctcgttcctgtccgtcaccattgacgccaacctggccacggacccgcggttcctcatcctcctgggttctccaaagcttcgtaccttggccagaggcttgtctcctgcgtacctgaggtttggtggcaccaagacagacttcctaattttcgatcccaagaaggaatcaacctttgaagagagaagttactggggttcaggggcaggatccggcgccgaatggccctaccaggagcaattgctactccgagaacactaccagaaaaagttcaagaacagcacctactcaagaagctctgtagatgtgctatacacttttgcaaactgctcaggactggacttgatctttggcctaaatgcgttattaagaacagcagatttgcagtggaacagttctaatgctcagttgctcctggactactgctcttccaaggggtataacatttcttgggaactaggcaatgaacctaacagtttccttaagaaggctgatattttcatcaatgggtcgcagttaggagaagattttattcaattgcataaacttctaagaaagtccaccttcaaaaatgcaaaactctatggtcctgatgttggtcagcctcgaagaaagacggctaagatgctgaagagcttcctgaaggctggtggagaagtgattgattcagttacatggcatcactactatttgaatggacggactgctaccagggaagattttctaaaccctgatgtattggacatttttatttcatctgtgcaaaaagttttccaggtggttgagagcaccaggcctggcaagaaggtctggttaggagaaacaagctctgcatatggaggcggagcgcccttgctatccgacacctttgcagctggctttatgtggctggataaattgggcctgtcagcccgaatgggaatagaagtggtgatgaggcaagtattctttggagcaggaaactaccatttagtggatgaaaacttcgatcctttacctgattattggctatctcttctgttcaagaaattggtgggcaccaaggtgttaatggcaagcgtgcaaggttcaaagagaaggaagcttcgagtataccttcattgcacaaacactgacaatccaaggtataaagaaggagatttaactctgtatgccataaacctccataatgtcaccaagtacttgcggttaccctatcctttttctaacaagcaagtggataaataccttctaagacctttgggacctcatggattactttccaaatctgtccaactcaatggtctaactctaaagatggtggatgatcaaaccttgccacctttaatggaaaaacctctccggccaggaagttcactgggcttgccagctttctcatatagtttttttgtgataagaaatgccaaagttgctgcttgcatctga

SEQ ID NO: 24 - DNA, 1524 bp, Artificial Sequence
  hep GS6
atgctgctgcgctcgaagcctgcgctgccgccgccgctgatgctgctgctcctggggccgctgggtcccctctcccctggcgccctgccccgacctgcgcaagcacaggacgtcgtggacctggacttcttcacccaggagccgctgcacctggtgagcccctcgttcctgtccgtcaccattgacgccaacctggccacggacccgcggttcctcatcctcctgggttctccaaagcttcgtaccttggccagaggcttgtctcctgcgtacctgaggtttggtggcaccaagacagacttcctaattttcgatcccaagaaggaaggtagcggttccggctctggtagcggctctggtagcaaaaagttcaagaacagcacctactcaagaagctctgtagatgtgctatacacttttgcaaactgctcaggactggacttgatctttggcctaaatgcgttattaagaacagcagatttgcagtggaacagttctaatgctcagttgctcctggactactgctcttccaaggggtataacatttcttgggaactaggcaatgaacctaacagtttccttaagaaggctgatattttcatcaatgggtcgcagttaggagaagattttattcaattgcataaacttctaagaaagtccaccttcaaaaatgcaaaactctatggtcctgatgttggtcagcctcgaagaaagacggctaagatgctgaagagcttcctgaaggctggtggagaagtgattgattcagttacatggcatcactactatttgaatggacggactgctaccagggaagattttctaaaccctgatgtattggacatttttatttcatctgtgcaaaaagttttccaggtggttgagagcaccaggcctggcaagaaggtctggttaggagaaacaagctctgcatatggaggcggagcgcccttgctatccgacacctttgcagctggctttatgtggctggataaattgggcctgtcagcccgaatgggaatagaagtggtgatgaggcaagtattctttggagcaggaaactaccatttagtggatgaaaacttcgatcctttacctgattattggctatctcttctgttcaagaaattggtgggcaccaaggtgttaatggcaagcgtgcaaggttcaaagagaaggaagcttcgagtataccttcattgcacaaacactgacaatccaaggtataaagaaggagatttaactctgtatgccataaacctccataatgtcaccaagtacttgcggttaccctatcctttttctaacaagcaagtggataaataccttctaagacctttgggacctcatggattactttccaaatctgtccaactcaatggtctaactctaaagatggtggatgatcaaaccttgccacctttaatggaaaaacctctccggccaggaagttcactgggcttgccagctttctcatatagtttttttgtgataagaaatgccaaagttgctgcttgcatctga

SEQ ID NO: 25 - protein, 527 aa, Artificial Sequence
  hep GS-A4
MLLRSKPALPPPLMLLLLGPLGPLSPGALPRPAQAQDVVDLDFFTQEPLHLVSPSFLSVTIDANLATDPRFLILLGSPKLRTLARGLSPAYLRFGGTKTDFLIFDPKKESTFEERSYWGSGAGSGAEWPYQEQLLLREHYQKKFKNSTYSRSSVDVLYTFANCSGLDLIFGLNALLRTADLQWNSSNAQLLLDYCSSKGYNISWELGNEPNSFLKKADIFINGSQLGEDFIQLHKLLRKSTFKNAKLYGPDVGQPRRKTAKMLKSFLKAGGEVIDSVTWHHYYLNGRTATREDFLNPDVLDIFISSVQKVFQVVESTRPGKKVWLGETSSAYGGGAPLLSDTFAAGFMWLDKLGLSARMGIEVVMRQVFFGAGNYHLVDENFDPLPDYWLSLLFKKLVGTKVLMASVQGSKRRKLRVYLHCTNTDNPRYKEGDLTLYAINLHNVTKYLRLPYPFSNKQVDKYLLRPLGPHGLLSKSVQLNGLTLKMVDDQTLPPLMEKPLRPGSSLGLPAFSYSFFVIRNAKVAACI

SEQ ID NO: 26 - protein, 507 aa, Artificial Sequence
  hep GS-A6
MLLRSKPALPPPLMLLLLGPLGPLSPGALPRPAQAQDVVDLDFFTQEPLHLVSPSFLSVTIDANLATDPRFLILLGSPKLRTLARGLSPAYLRFGGTKTDFLIFDPKKEGSGSGSGSGSGSKKFKNSTYSRSSVDVLYTFANCSGLDLIFGLNALLRTADLQWNSSNAQLLLDYCSSKGYNISWELGNEPNSFLKKADIFINGSQLGEDFIQLHKLLRKSTFKNAKLYGPDVGQPRRKTAKMLKSFLKAGGEVIDSVTWHHYYLNGRTATREDFLNPDVLDIFISSVQKVFQVVESTRPGKKVWLGETSSAYGGGAPLLSDTFAAGFMWLDKLGLSARMGIEVVMRQVFFGAGNYHLVDENFDPLPDYWLSLLFKKLVGTKVLMASVQGSKRRKLRVYLHCTNTDNPRYKEGDLTLYAINLHNVTKYLRLPYPFSNKQVDKYLLRPLGPHGLLSKSVQLNGLTLKMVDDQTLPPLMEKPLRPGSSLGLPAFSYSFFVIRNAKVAACI

SEQ ID NO: 27 - protein, 526 aa, Artificial Sequence
  hep Hya1
MLLRSKPALPPPLMLLLLGPLGPLSPGALPRPAQAQDVVDLDFFTQEPLHLVSPSFLSVTIDANLATDPRFLILLGSPKLRTLARGLSPAYLRFGGTKTDFLIFDPKKESTFEERSYWAFKDKTPEWPYQEQLLLREHYQKKFKNSTYSRSSVDVLYTFANCSGLDLIFGLNALLRTADLQWNSSNAQLLLDYCSSKGYNISWELGNEPNSFLKKADIFINGSQLGEDFIQLHKLLRKSTFKNAKLYGPDVGQPRRKTAKMLKSFLKAGGEVIDSVTWHHYYLNGRTATREDFLNPDVLDIFISSVQKVFQVVESTRPGKKVWLGETSSAYGGGAPLLSDTFAAGFMWLDKLGLSARMGIEVVMRQVFFGAGNYHLVDENFDPLPDYWLSLLFKKLVGTKVLMASVQGSKRRKLRVYLHCTNTDNPRYKEGDLTLYAINLHNVTKYLRLPYPFSNKQVDKYLLRPLGPHGLLSKSVQLNGLTLKMVDDQTLPPLMEKPLRPGSSLGLPAFSYSFFVIRNAKVAACI

SEQ ID NO: 28 - DNA, 1581 bp, Artificial Sequence
  hep Hya1
atgctgctgcgctcgaagcctgcgctgccgccgccgctgatgctgctgctcctggggccgctgggtcccctctcccctggcgccctgccccgacctgcgcaagcacaggacgtcgtggacctggacttcttcacccaggagccgctgcacctggtgagcccctcgttcctgtccgtcaccattgacgccaacctggccacggacccgcggttcctcatcctcctgggttctccaaagcttcgtaccttggccagaggcttgtctcctgcgtacctgaggtttggtggcaccaagacagacttcctaattttcgatcccaagaaggaatcaacctttgaagagagaagttactgggccttcaaggacaagacccccgaatggccctaccaggagcaattgctactccgagaacactaccagaaaaagttcaagaacagcacctactcaagaagctctgtagatgtgctatacacttttgcaaactgctcaggactggacttgatctttggcctaaatgcgttattaagaacagcagatttgcagtggaacagttctaatgctcagttgctcctggactactgctcttccaaggggtataacatttcttgggaactaggcaatgaacctaacagtttccttaagaaggctgatattttcatcaatgggtcgcagttaggagaagattttattcaattgcataaacttctaagaaagtccaccttcaaaaatgcaaaactctatggtcctgatgttggtcagcctcgaagaaagacggctaagatgctgaagagcttcctgaaggctggtggagaagtgattgattcagttacatggcatcactactatttgaatggacggactgctaccagggaagattttctaaaccctgatgtattggacatttttatttcatctgtgcaaaaagttttccaggtggttgagagcaccaggcctggcaagaaggtctggttaggagaaacaagctctgcatatggaggcggagcgcccttgctatccgacacctttgcagctggctttatgtggctggataaattgggcctgtcagcccgaatgggaatagaagtggtgatgaggcaagtattctttggagcaggaaactaccatttagtggatgaaaacttcgatcctttacctgattattggctatctcttctgttcaagaaattggtgggcaccaaggtgttaatggcaagcgtgcaaggttcaaagagaaggaagcttcgagtataccttcattgcacaaacactgacaatccaaggtataaagaaggagatttaactctgtatgccataaacctccataatgtcaccaagtacttgcggttaccctatcctttttctaacaagcaagtggataaataccttctaagacctttgggacctcatggattactttccaaatctgtccaactcaatggtctaactctaaagatggtggatgatcaaaccttgccacctttaatggaaaaacctctccggccaggaagttcactgggcttgccagctttctcatatagtttttttgtgataagaaatgccaaagttgctgcttgcatctga

SEQ ID NO: 29 - protein, 570 aa, Artificial Sequence
  hep TEV110-158
MLLRSKPALPPPLMLLLLGPLGPLSPGALPRPAQAQDVVDLDFFTQEPLHLVSPSFLSVTIDANLATDPRFLILLGSPKLRTLARGLSPAYLRFGGTKTDFLIFDPKKEGSGSENLYFQGSGSTFEERSYWQSQVNQDICKYGSIPPDVEEKLRLEWPYQEQLLLREHYQSGSGENLYFQGSGSKKFKNSTYSRSSVDVLYTFANCSGLDLIFGLNALLRTADLQWNSSNAQLLLDYCSSKGYNISWELGNEPNSFLKKADIFINGSQLGEDFIQLHKLLRKSTFKNAKLYGPDVGQPRRKTAKMLKSFLKAGGEVIDSVTWHHYYLNGRTATREDFLNPDVLDIFISSVQKVFQVVESTRPGKKVWLGETSSAYGGGAPLLSDTFAAGFMWLDKLGLSARMGIEVVMRQVFFGAGNYHLVDENFDPLPDYWLSLLFKKLVGTKVLMASVQGSKRRKLRVYLHCTNTDNPRYKEGDLTLYAINLHNVTKYLRLPYPFSNKQVDKYLLRPLGPHGLLSKSVQLNGLTLKMVDDQTLPPLMEKPLRPGSSLGLPAFSYSFFVIRNAKVAACI

SEQ ID NO: 30 - DNA, 1668 bp, Artificial Sequence
  hep TEV110
atgctgctgcgctcgaagcctgcgctgccgccgccgctgatgctgctgctcctggggccgctgggtcccctctcccctggcgccctgccccgacctgcgcaagcacaggacgtcgtggacctggacttcttcacccaggagccgctgcacctggtgagcccctcgttcctgtccgtcaccattgacgccaacctggccacggacccgcggttcctcatcctcctgggttctccaaagcttcgtaccttggccagaggcttgtctcctgcgtacctgaggtttggtggcaccaagacagacttcctaattttcgatcccaagaaggaaggcagcggatctgagaacctgtacttccagggttccggttcaacctttgaagagagaagttactggcaatctcaagtcaaccaggatatttgcaaatatggatccatccctcctgatgtggaggagaagttacggttggaatggccctaccaggagcaattgctactccgagaacactaccagaaaaagttcaagaacagcacctactcaagaagctctgtagatgtgctatacacttttgcaaactgctcaggactggacttgatctttggcctaaatgcgttattaagaacagcagatttgcagtggaacagttctaatgctcagttgctcctggactactgctcttccaaggggtataacatttcttgggaactaggcaatgaacctaacagtttccttaagaaggctgatattttcatcaatgggtcgcagttaggagaagattttattcaattgcataaacttctaagaaagtccaccttcaaaaatgcaaaactctatggtcctgatgttggtcagcctcgaagaaagacggctaagatgctgaagagcttcctgaaggctggtggagaagtgattgattcagttacatggcatcactactatttgaatggacggactgctaccagggaagattttctaaaccctgatgtattggacatttttatttcatctgtgcaaaaagttttccaggtggttgagagcaccaggcctggcaagaaggtctggttaggagaaacaagctctgcatatggaggcggagcgcccttgctatccgacacctttgcagctggctttatgtggctggataaattgggcctgtcagcccgaatgggaatagaagtggtgatgaggcaagtattctttggagcaggaaactaccatttagtggatgaaaacttcgatcctttacctgattattggctatctcttctgttcaagaaattggtgggcaccaaggtgttaatggcaagcgtgcaaggttcaaagagaaggaagcttcgagtataccttcattgcacaaacactgacaatccaaggtataaagaaggagatttaactctgtatgccataaacctccataatgtcaccaagtacttgcggttaccctatcctttttctaacaagcaagtggataaataccttctaagacctttgggacctcatggattactttccaaatctgtccaactcaatggtctaactctaaagatggtggatgatcaaaccttgccacctttaatggaaaaacctctccggccaggaagttcactgggcttgccagctttctcatatagtttttttgtgataagaaatgccaaagttgctgcttgcatc

SEQ ID NO: 31 - protein, 556 aa, Artificial Sequence
  hep TEV110
MLLRSKPALPPPLMLLLLGPLGPLSPGALPRPAQAQDVVDLDFFTQEPLHLVSPSFLSVTIDANLATDPRFLILLGSPKLRTLARGLSPAYLRFGGTKTDFLIFDPKKEGSGSENLYFQGSGSTFEERSYWQSQVNQDICKYGSIPPDVEEKLRLEWPYQEQLLLREHYQKKFKNSTYSRSSVDVLYTFANCSGLDLIFGLNALLRTADLQWNSSNAQLLLDYCSSKGYNISWELGNEPNSFLKKADIFINGSQLGEDFIQLHKLLRKSTFKNAKLYGPDVGQPRRKTAKMLKSFLKAGGEVIDSVTWHHYYLNGRTATREDFLNPDVLDIFISSVQKVFQVVESTRPGKKVWLGETSSAYGGGAPLLSDTFAAGFMWLDKLGLSARMGIEVVMRQVFFGAGNYHLVDENFDPLPDYWLSLLFKKLVGTKVLMASVQGSKRRKLRVYLHCTNTDNPRYKEGDLTLYAINLHNVTKYLRLPYPFSNKQVDKYLLRPLGPHGLLSKSVQLNGLTLKMVDDQTLPPLMEKPLRPGSSLGLPAFSYSFFVIRNAKVAACI

SEQ ID NO: 32 - DNA, 1710 bp, Artificial Sequence
  hep TEV110/158
atgctgctgcgctcgaagcctgcgctgccgccgccgctgatgctgctgctcctggggccgctgggtcccctctcccctggcgccctgccccgacctgcgcaagcacaggacgtcgtggacctggacttcttcacccaggagccgctgcacctggtgagcccctcgttcctgtccgtcaccattgacgccaacctggccacggacccgcggttcctcatcctcctgggttctccaaagcttcgtaccttggccagaggcttgtctcctgcgtacctgaggtttggtggcaccaagacagacttcctaattttcgatcccaagaaggaaggcagcggatctgagaacctgtacttccagggttccggttcaacctttgaagagagaagttactggcaatctcaagtcaaccaggatatttgcaaatatggatccatccctcctgatgtggaggagaagttacggttggaatggccctaccaggagcaattgctactccgagaacactaccagtctggatccggtgaaaatctctattttcagggctcaggaagtaaaaagttcaagaacagcacctactcaagaagctctgtagatgtgctatacacttttgcaaactgctcaggactggacttgatctttggcctaaatgcgttattaagaacagcagatttgcagtggaacagttctaatgctcagttgctcctggactactgctcttccaaggggtataacatttcttgggaactaggcaatgaacctaacagtttccttaagaaggctgatattttcatcaatgggtcgcagttaggagaagattttattcaattgcataaacttctaagaaagtccaccttcaaaaatgcaaaactctatggtcctgatgttggtcagcctcgaagaaagacggctaagatgctgaagagcttcctgaaggctggtggagaagtgattgattcagttacatggcatcactactatttgaatggacggactgctaccagggaagattttctaaaccctgatgtattggacatttttatttcatctgtgcaaaaagttttccaggtggttgagagcaccaggcctggcaagaaggtctggttaggagaaacaagctctgcatatggaggcggagcgcccttgctatccgacacctttgcagctggctttatgtggctggataaattgggcctgtcagcccgaatgggaatagaagtggtgatgaggcaagtattctttggagcaggaaactaccatttagtggatgaaaacttcgatcctttacctgattattggctatctcttctgttcaagaaattggtgggcaccaaggtgttaatggcaagcgtgcaaggttcaaagagaaggaagcttcgagtataccttcattgcacaaacactgacaatccaaggtataaagaaggagatttaactctgtatgccataaacctccataatgtcaccaagtacttgcggttaccctatcctttttctaacaagcaagtggataaataccttctaagacctttgggacctcatggattactttccaaatctgtccaactcaatggtctaactctaaagatggtggatgatcaaaccttgccacctttaatggaaaaacctctccggccaggaagttcactgggcttgccagctttctcatatagtttttttgtgataagaaatgccaaagttgctgcttgcatc

SEQ ID NO: 33 - protein, 174 aa, Homo Sapiens
QQDVVDLDFFTQEPLHLVSPSFLSVTIDANLATDPRFLILLGSPKLRTLARGLSPAYLRFGGTKTDFLIFDPKKESTFEERSYWQSQVNQDICKYGSIPPDVEEKLRLEWPYQEQLLLREHYQKKFKNSTYSRSSVDVLYTFANCSGLDLIFGLNALLRTADLQWNSSNAQLLL

SEQ ID NO: 34 - protein, 174 aa, Mus Musculus
TDDVVDLEFYTKRPLRSVSPSFLSITIDASLATDPRFLTFLGSPRLRALARGLSPAYLRFGGTKTDFLIFDPDKEPTSEERSYWKSQVNHDICRSEPVSAAVLRKLQVEWPFQELLLLREQYQKEFKNSTYSRSSVDMLYSFAKCSGLDLIFGLNALLRTPDLRWNSSNAQLLL

SEQ ID NO: 35 - protein, 174 aa, Bos taurus
ADDAAELEFFTERPLHLVSPAFLSFTIDANLATDPRFFTFLGSSKLRTLARGLAPAYLRFGGNKGDFLIFDPKKEPAFEERSYWLSQSNQDICKSGSIPSDVEEKLRLEWPFQEQVLLREQYQKKFTNSTYSRSSVDMLYTFASCSGLNLIFGVNALLRTTDMHWDSSNAQLLL

SEQ ID NO: 36 - protein, 173 aa, Gallus Gallus
PRRTAELQLGLREPIGAVSPAFLSLTLDASLARDPRFVALLRHPKLHTLASGLSPGFLRFGGTSTDFLIFNPNKDSTWEEKVLSEFQAKDVCEAWPSFAVVPKLLLTQWPLQEKLLLAEHSWKKHKNTTITRSTLDILHTFASSSGFRLVFGLNALLRRAGLQWDSSNAKQLL

SEQ ID NO: 37 - protein, 189 aa, Homo Sapien
EKTLILLDVSTKNPVRTVNENFLSLQLDPSIIHDGWLDFLSSKRLVTLARGLSPAFLRFGGKRTDFLQFQNLRNPAKSRGGPGPDYYLKNYEDDIVRSDVALDKQKGCKIAQHPDVMLELQREKAAQMHLVLLKEQFSNTYSNLILTARSLDKLYNSADCSGLHLIFALNALRRNPNNSWNSSSALSLL

SEQ ID NO: 38 - protein, 151 aa, Bombyx Mori
VRYFVTINENQEDIKLISEDFLSFGIDTIEIENYNRINYSDTRLRELAAALSPARLRLGGTMSERLIFSKENIPISCHNCSYKSYPKSLCQLIEKPCKHKHKFLPFFIMTGNEWNQINDFCRKTNLKLLFSLNAMLRDNHGWNEKNARELI

SEQ ID NO: 39 - protein, 147 aa, Hirudinaria manillensis
KNVIASVSESFHGVAFDASLFSPKGPWSFVNITSPKLFKLLEGLSPGYFRVGGTFANWLFFDLDENNKWKDYWAFKDKTPETATITRRWLFRKQNNLKKETFDDLVKLTKGSKMRLLFDLNAEVRTGYEIGKKTTSTWDSSEAEKLF

SEQ ID NO: 40 - protein, 150 aa, Scutellaria baicallensis
NYVCATLDLWPPTKCNYGNCPWGKSSFLNLDLNNNIIRNAVKEFAPLKLRFGGTLQDRLVYQTSRDEPCDSTFYNNTNLILDFSHACLSLDRWDEINQFILETGSEAVFGLNALRGKTVEIKGIIKDGQYLGETTTAVGEWDYSNSKFLI

SEQ ID NO: 41 - protein, 138 aa, Arabidopsis thaliana
NFVCATLDWWPHDKCNYDQCPWGYSSVINMDLTRPLLTKAIKAFKPLRIRIGGSLQDQVIYDVGNLKTPCRPFQKMNSGLFGFSKGCLHMKRWDELNSFLTATGAVVTFGLNALRGRHKLRGKAWGGAWDHINTQDFL

SEQ ID NO: 42 - protein, 138 aa, Arabidopsis thaliana
DFICATLDWWPPEKCDYGSCSWDHASILNLDLNNVILQNAIKAFAPLKIRIGGTLQDIVIYETPDSKQPCLPFTKNSSILFGYTQGCLPMRRWDELNAFFRKTGTKVIFGLNALSGRSIKSNGEAIGAWNYTNAESFI

SEQ ID NO: 43 - protein, 138 aa, Arabidopsis thaliana
NFICATLDWWPPEKCNYDQCPWGYASLINLNLASPLLAKAIQAFRTLRIRIGGSLQDQVIYDVGDLKTPCTQFKKTDDGLFGFSEGCLYMKRWDEVNHFFNATGAIVTFGLNALHGRNKLNGTAWGGDWDHTNTQDFM

SEQ ID NO: 44 - protein, 7 aa, Artificial Sequence
  Peptide
AFKDKTP